SEQUENCE LISTING (80 sequences)

SEQ ID NO: 1 - protein, 366 aa, Homo sapiens
  MISC_FEATURE (1)..(4): optionally absent
DAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 2 - protein, 522 aa, Homo sapiens
  MISC_FEATURE (1)..(1): optionally absent
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 3 - protein, 362 aa, Mus musculus
  MISC_FEATURE (1)..(3): optionally absent
SPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 4 - protein, 506 aa, Mus musculus
  MISC_FEATURE (1)..(1): optionally absent
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 5 - protein, 366 aa, Artificial Sequence
  Synthetic
DAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRMDCLMLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 6 - protein, 522 aa, Artificial Sequence
  Synthetic
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRMDCLMLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 7 - protein, 362 aa, Artificial Sequence
  Synthetic
SPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 8 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 9 - protein, 366 aa, Artificial Sequence
  Synthetic
DAAPGASKLRAVLEKLKLDPDDISTAAGMVKGVVDHLLLRLKCDSAFRGVGLLPVGSFYEKVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 10 - protein, 366 aa, Artificial Sequence
  Synthetic
DAAPGASKLRAVLEKLKLDPDDISTAAGMVKGVVDHLLLRLKCDSAFRGVGLLLVGSFYEKVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 11 - protein, 366 aa, Artificial Sequence
  Synthetic
DAAPGASKLRAVLEKLKLDPDDISTAAGMVKGVVDHLLLRLKCDSAFRGVGLLLTGSFYEKVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 12 - protein, 366 aa, Artificial Sequence
  Synthetic
DAAPGASKLRAVLEKLKLDPDDISTAAGMVKGVVDHLLLRLKCDSAFRGVGLLFVGSIYEKVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 13 - protein, 522 aa, Artificial Sequence
  Synthetic
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLDPDDISTAAGMVKGVVDHLLLRLKCDSAFRGVGLLPVGSFYEKVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 14 - protein, 522 aa, Artificial Sequence
  Synthetic
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLDPDDISTAAGMVKGVVDHLLLRLKCDSAFRGVGLLLVGSFYEKVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 15 - protein, 522 aa, Artificial Sequence
  Synthetic
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLDPDDISTAAGMVKGVVDHLLLRLKCDSAFRGVGLLLTGSFYEKVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 16 - protein, 522 aa, Artificial Sequence
  Synthetic
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLDPDDISTAAGMVKGVVDHLLLRLKCDSAFRGVGLLFVGSIYEKVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 17 - protein, 362 aa, Artificial Sequence
  Synthetic
SPDKLKKVLDKLRLDPKDISEAAETVNKVVERLLRRMQKRESEFKGVEQLPVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 18 - protein, 362 aa, Artificial Sequence
  Synthetic
SPDKLKKVLDKLRLDPKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 19 - protein, 362 aa, Artificial Sequence
  Synthetic
SPDKLKKVLDKLRLDPKDISEAAETVNKVVERLLRRMQKRESEFKGVEQLLTGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 20 - protein, 354 aa, Artificial Sequence
  Synthetic
SPDKLKKVLDKLRLDPKDISEAAETVNKVVERLLRRMQKRESEFKGVEQLFVGSIYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNN

SEQ ID NO: 21 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 22 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLPVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 23 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 24 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLTGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 25 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLFVGSIYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 26 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 27 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKPKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 28 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDRKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 29 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRIPAKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 30 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLPAKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 31 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLPRKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 32 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLSLDPKDISEAAETVNKVVERLLRRMQKRESEFKGVEQLLVGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 33 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLIGSFYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 34 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLVGSIYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 35 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLIGSIYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 36 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLVGSFYEKVKIGAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 37 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLVGSFYEKVKIIAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 38 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLVGSFYEKVKIVAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 39 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYEEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 40 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYFEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 41 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYWEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 42 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYEEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 43 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYFEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 44 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYWEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 45 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLNTGSYEEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 46 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLNTGSYFEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 47 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLNTGSYWEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 48 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLPVGSFEEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 49 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLPVGSFFEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 50 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLPVGSFWEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 51 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLVGSFEEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 52 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLVGSFFEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 53 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLVGSFWEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 54 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLTGSFEEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 55 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLTGSFFEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 56 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLLTGSFWEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 57 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLFVGSIEEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 58 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLFVGSIFEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 59 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLDPKDISEAAETVNKVVERLLRRMQKRESEFKGVEQLFVGSIWEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 60 - DNA, 27 bp, Artificial Sequence
  Synthetic
gaacagaaactgattagcgaagaagat

SEQ ID NO: 61 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 62 - protein, 362 aa, Artificial Sequence; identical to SEQ ID NO: 3
  Synthetic

SEQ ID NO: 63 - protein, 522 aa, Artificial Sequence; identical to SEQ ID NO: 2
  Synthetic

SEQ ID NO: 64 - protein, 507 aa, Artificial Sequence; identical to SEQ ID NO: 8
  Synthetic

SEQ ID NO: 65 - protein, 362 aa, Artificial Sequence; identical to SEQ ID NO: 7
  Synthetic

SEQ ID NO: 66 - protein, 522 aa, Artificial Sequence; identical to SEQ ID NO: 6
  Synthetic

SEQ ID NO: 67 - protein, 507 aa, Artificial Sequence
  Synthetic
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRAECLALMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 68 - protein, 507 aa, Artificial Sequence; identical to SEQ ID NO: 21
  Synthetic

SEQ ID NO: 69 - protein, 507 aa, Artificial Sequence; identical to SEQ ID NO: 22
  Synthetic

SEQ ID NO: 70 - protein, 362 aa, Artificial Sequence; identical to SEQ ID NO: 17
  Synthetic

SEQ ID NO: 71 - protein, 522 aa, Artificial Sequence; identical to SEQ ID NO: 13
  Synthetic

SEQ ID NO: 72 - protein, 507 aa, Artificial Sequence; identical to SEQ ID NO: 23
  Synthetic

SEQ ID NO: 73 - protein, 362 aa, Artificial Sequence; identical to SEQ ID NO: 18
  Synthetic

SEQ ID NO: 74 - protein, 522 aa, Artificial Sequence; identical to SEQ ID NO: 14
  Synthetic

SEQ ID NO: 75 - protein, 507 aa, Artificial Sequence; identical to SEQ ID NO: 24
  Synthetic

SEQ ID NO: 76 - protein, 362 aa, Artificial Sequence; identical to SEQ ID NO: 19
  Synthetic

SEQ ID NO: 77 - protein, 522 aa, Artificial Sequence; identical to SEQ ID NO: 15
  Synthetic

SEQ ID NO: 78 - protein, 507 aa, Artificial Sequence; identical to SEQ ID NO: 25
  Synthetic

SEQ ID NO: 79 - protein, 362 aa, Artificial Sequence
  Synthetic
SPDKLKKVLDKLRLDPKDISEAAETVNKVVERLLRRMQKRESEFKGVEQLFVGSIYEKVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRMECLMLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 80 - protein, 522 aa, Artificial Sequence; identical to SEQ ID NO: 16
  Synthetic

(19) United States
(12) Patent Application Publication
(10) Pub. No.: US 2024/0252598 A1
(43) Pub. Date: Aug. 1, 2024

(54) DESIGNED CONSTITUTIVELY ACTIVE CYCLIC GMP-AMP SYNTHASE AS A GENETICALLY ENCODED STIMULANT OF INTERFERONS

(71) Applicant: University of Washington, Seattle, WA (US)

(72) Inventors: Quinton M. DOWLING, Seattle, WA (US); Neil P. KING, Seattle, WA (US); Daniel Brewster STETSON, Seattle, WA (US); Hannah VOLKMAN, Seattle, WA (US); Elizabeth Erin GRAY, Seattle, WA (US)

(21) Appl. No.: 18/549,539

(22) PCT Filed: Apr. 11, 2022
(86) PCT No.: PCT/US2022/024255

§ 371 Date: Sep. 7, 2023

Related U.S. Application Data
(60) Provisional application No. 63/174,279, filed on Apr. 13, 2021.

(51) Int. Cl. A61K 38/45; C12N 9/12

(52) U.S. Cl. CPC A61K 38/45; C12N 9/1241; C12Y 207/07

(57) ABSTRACT

The disclosure provides constitutively active Cyclic GMP-AMP Synthase (cGAS) polypeptide mutants and. methods for their use as adjuvants.

### CROSS REFERENCE

This application claims priority to U.S. Provisional Application Ser. No. 63/174,279 filed Apr. 13, 2021, incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Apr. 11, 2022 having the file name “21-0379-WO-SeqList_ST25.txt” and is 322 kb in size.

### BACKGROUND

Cyclic GMP-AMP Synthase (cGAS) is a pattern recognition receptor critical for the innate immune response to intracellular pathogens, DNA damage, tumorigenesis, and senescence. Binding to double-stranded DNA (dsDNA) induces conformational changes in cGAS that activate the enzyme to produce 2′-3′ cyclic GMP-AMP (cGAMP), a second messenger that initiates a potent interferon response through its receptor, STING. The cGAS-STING pathway is a major target for prevention and treatment of infectious disease, cancer, and autoimmunity, although most current efforts are limited to small molecule drugs.

### SUMMARY

In one aspect, the disclosure provides polypeptides comprising an amino acid sequence at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:1 or 2, wherein the polypeptide is a constitutively active Cyclic GMP-AMP Synthase (cGAS) mutant;
wherein residues in parentheses are optional and may be present or absent;
wherein relative to residue numbering in SEQ ID NO:1:
residue 56 is G, residue 57 is S, residue 60 is E, residue 69 is E, and residue 71 is D; and
wherein one or both of the following are true:
(a) residue 19 is P and residue 20 is A; or residue 19 is D and residue 20 is P; and
(b) residue 54 is P, L, or F and residue 58 is F, I, W, or L.

In various aspects, residue 55 is V, I, A, S, or G relative to residue numbering in SEQ ID NO:1, preferably wherein residue 55 is V or I, most preferably wherein residue 55 is V; residue 61 is K relative to residue numbering in SEQ ID NO:1; 1, 2, or all 3 of the following are true: residue 62 is V, residue 63 is K, and/or residue 64 is I, relative to residue numbering in SEQ ID NO:1; and/or one or more of the following is true relative to residue numbering in SEQ ID NO: 1:
residue 17 is S;
residue 18 is I;
residue 59 is E, F, W, L, or I; preferably wherein residue 59 is F. W, L, or I, more preferably wherein residue 59 is F or W;
residue 65 is G or V;
residue 251 is M; and/or
residue 255 is M.

In another aspect, the disclosure provides polypeptides comprising an amino acid sequence at least 20%, 25%, 30%, 35%, 40%, 45%, 50%, 55% 60%, 65%, 70%, 75%, 80%, 85%, 90%, 91%, 92%, 93%, 94%, 95%, 96%, 97%, 98%, or 99% identical to the amino acid sequence of SEQ ID NO:3 or 4,
wherein the polypeptide is a constitutively active Cyclic GMP-AMP Synthase (cGAS) mutant;
wherein residues in parentheses are optional and may be present or absent;
wherein relative to residue numbering in SEQ ID NO:3:
residue 53 is G, residue 54 is S, residue 57 is E, residue 66 is E, and residue 68 is D; and
wherein one or both of the following are true:
(a) residue 15 is P and residue 16 is A; or residue 15 is D and residue 16 is P; and
(b) residue 51 is P, L, or F and residue 55 is F, I, W, or L.

In various embodiments, residue 51 is P, L, or F and residue 55 is F, I, W, or L, relative to residue numbering in SEQ ID NO:3; residue 52 is V, I, A, G, or S relative to residue numbering in SEQ ID NO:3; preferably wherein residue 52 is V or I, or more preferably wherein residue 52 is V; residue 58 is K relative to residue numbering in SEQ ID NO:3; 1, 2, or all 3 are true: residue 59 is V, residue 60 is K, and/or residue 61 is I relative to residue numbering in SEQ ID NO:3; and/or one or more of the following is true relative to residue numbering in SEQ ID NO:3:
residue 13 is S;
residue 14 is I;
residue 56 is E, F, W, L, or I; preferably wherein residue 56 is F. W, L, or I, more preferably wherein residue 56 is F ow W;
residue 62 is G or V;
residue 250 is M;
residue 254 is M.

In various embodiments, the polypeptide does not include the mutation or combination of mutations listed on a single line in Table 1 relative to residue numbering in SEQ ID NO:1, in Table 2 relative to residue numbering in SEQ ID NO:2, in Table 3 relative to residue numbering in SEQ ID NO:3, or in Table 4 relative to residue numbering in SEQ ID NO:4.

In other embodiments, the polypeptides comprise an amino acid sequence at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:5-59.

In other aspects, the disclosure provides nucleic acids encoding the polypeptide of any embodiment of the disclosure; expression vectors comprising the nucleic acid operatively linked to a suitable control sequence; host cell comprising the polypeptide, composition, nucleic acid, or expression vector of any embodiment herein, pharmaceutical compositions comprising the nucleic acid, expression vector, or host cell of any embodiment herein, and a pharmaceutically acceptable carrier; and methods and uses of the polypeptides, compositions, nucleic acids, expression vectors, or host cells of any embodiment herein for any therapeutic use, including but not limited for use as an adjuvant, such as for use as an adjuvant in combination with a prophylactic or therapeutic vaccine.

### DESCRIPTION OF THE FIGURES

FIG. 1(a-g). Computational design of a DNA-independent constitutively active cGAS. a, cGAS is activated by binding to dsDNA (inactive PDB ID 4K8V (1), active PDB ID 5N61 (15)). b, Conformational changes that occur upon dsDNA binding provide an opportunity to use computational design to stabilize the active conformation in the absence of dsDNA. Local changes are observed in the N-terminal helix (region 1) and active site loop (region 2), and a more global change brings the NTase core and C-domain closer together (region 3). c, A two-state design approach for generating CA-cGAS variants destabilizes the inactive conformation and stabilizes the active conformation in the absence of dsDNA. d, The residue-residue distance difference matrix of the active (PDB ID 4K97) and inactive (PDB ID 4K8V) conformations of mcGAS highlights two regions (1 and 2) to target for design. The matrix shows pairs of contacting residues (off-diagonal points) that are closer together or further apart in the active conformation compared to the inactive. e, Enumeration of all possible point mutants in regions 1 and 2. The mutations are rank ordered by normalized Rosetta™ energy, calculated as the difference in the energetic impact of the mutation in the active and inactive states: (E_{mutant,active}−E_{WT,active})−(E_{mutant,inactive}−E_{WT,inactive}). f, Design models for representative high-ranking mutations in the inactive and active conformations. The rank order for these mutations is highlighted in E. g, Activity of CA-cGAS-04 compared to WT, K395A/399A, and K395M/K399M cGAS in cells, measured by ISRE assay. Bars represent the mean and error bars represent the standard deviation. The inset shows the design model for CA-cGAS-04, highlighting the two mutations added to the K395M/K399M background.

FIG. 2(a-d). Structural and bioinformatics comparison between cGAS and the static structural homolog MiD51. a, Full structure alignment between cGAS and MiD51. Boxed areas are shown in panel B. b, Local alignments of the boxed areas in panel A. Note that box 1 corresponds to cGAS region 1 and boxes 2 and 3 correspond to cGAS region 2. c, Bit scores for each member of a low stringency combined cGAS/MiD51 MSA compared to either the cGAS sequence (cGAS-Likeness) or MiD51 sequence (MiD51-Likeness). d, The difference in position-specific amino acid frequency (heatmap) in the boxed regions highlighted in FIG. 2b, residues 157-179 in box 1 and 196-210 in boxes 2 and 3. The bars show the scaled entropy difference at each position. Positions excluded because of substrate interactions marked with an asterisk; positions of interest in black.

FIG. 3(a-g). Activity of bioinformatics-guided CA-cGAS variants. a, Computational models of the activating mutations for each CA-cGAS variant in the active and inactive conformation. Note that CA-cGAS-22, -41, -42, and -50 also contain the CA-cGAS-04 mutations, which are not visible in this view. b, The activity of WT and K395M/K399M cGAS compared to CA-cGAS mutants as measured by ISRE assay 18 hours post transfection. c, The normalized fluorescence intensity of fATP changes over time as it reacts with GTP, catalyzed by WT cGAS (black) or CA-cGAS-50. The initial reaction rate is fit with a linear regression. d, Initial reaction rates for WT cGAS and CA-cGAS±dsDNA compared to CA-cGAS mutants without dsDNA. e, The effect of the presence of dsDNA on CA-cGAS activity. f, Salt dependence of WT cGAS and CA-cGAS-50 activity in the presence or absence of dsDNA. g, Activity of CA-cGAS-41 by ISRE assay after reverting one or both of the CA-cGAS-04 mutations to WT. In all plots bars represent the mean and error bars represent the standard deviation.

FIG. 4(a-g). In vivo activity of CA-cGAS-50. a, In vitro growth of CA-cGAS-50 B16-F10 cells with and without dox at 1 μg/ml. b, Schematic of study design. After implantation, tumors were allowed 10-11 days to establish before switching mice to chow containing dox. c, cGAMP concentration in tumor tissue 24 hours, 48 hours, and six days post CA-cGAS-50 induction (12, 13, and 17 days post tumor implantation respectively). For each timepoint, n=3 mice. d, Growth of CA-cGAS-50 B16-F10 tumors in STING^{-/-} mice. CA-cGAS-50 expression was induced by switching mice to dox chow on day 10 post tumor implantation. Note one datapoint is out of the displayed range at 16 days (shown in g). e, CA-cGAS-50 B16-F10 tumor volume in WT mice without dox chow treatment. f, CA-cGAS-50 B16-F10 tumor volume in WT mice with dox induction at day 10 post tumor implantation. g, Comparison of tumor volume of CA-cGAS-50-expressing tumors with and without dox induction in WT mice, or with dox induction in STING^{-/-} mice (n=15, 13, and 13 mice, respectively). Error bars represent SD. The box represents the median. upper, and lower quartile. Datapoints beyond the error bars are shown explicitly. Statistical analysis was performed using a two-way ANOVA with Tukey's correction for multiple comparisons. ns, not significant; *, p<0.5; **, p<0.01; ***, p<0.001; ****, p<0.001.

FIG. 5(a-d). Schematic of the ISRE assay. a, CA-cGAS plasmids are generated, combined with hSTING and ISRE-Luciferase plasmids, and b, transfected into 293T cells. c, Cells were incubated for 4-16 hours, lysed, and luciferase activity measured. d, Luciferase activity of the CA-cGAS-04 variant compared to WT, K395A/K399A, and K395M/K399M cGAS.

FIG. 6. Bifurcation in unique sequences used to find E-value cutoff. The number of sequences in multiple sequence alignments constructed using cGAS or MiD-51 input sequences, as well as the number of unique sequences found only in the cGAS or MiD51 alignment but not both, and the total number of sequences plotted as a function of the E-value cutoff used to generate the alignment. The ratio of unique sequences to total sequences is plotted on the secondary y axis.

FIG. 7(a-f). CA-cGAS activity, purification, and electrophoretic mobility shift. a, CA-cGAS activity by ISRE assay increases over time post transfection. b, Chromatogram of CA-cGAS purification by nickel affinity chromatography. The high absorbance at 260 nm relative to 280 nm indicates co-purification of significant amounts of nucleic acid. c, Chromatogram of CA-cGAS purification by heparin affinity chromatography monitored by absorbance at 280 nm and 260 nm. Note the much lower relative absorbance at 260 nm compared to (B). d, UV-vis spectrum of nickel-, heparin-, SEC-purified CA-cGAS and the ratio of absorbance to 260 nm to 280 nm, indicating pure protein. e, Agarose gel electrophoresis of dsDNA mixed with increasing concentrations of CA-cGAS. Top row stained with SYBR Safe. Bottom row is the same gel stained with coomassie. Molar ratios are, left to right, 1:0, 0:1, 1:0.1, 1:0.316, 1:1, 1:3.16, and 1:10 dsDNA to CA-cGAS. The increasing molar ratio is indicated by the triangle above the gel images. f, Activity in cells is not affected by truncating the unstructured N-terminal domain (compare to FIG. 3b).

FIG. 8(a-d). ISRE activity assays of additional mcGAS and hCGAS variants. a, The activity of bioinformatics consensus mutations applied to the break in the spine helix in a CA-cGAS-41 background. b, Based on the available activity data, computational, and bioinformatics analysis, we can infer a series of mutations that may enhance the activity of CA-cGAS. Specifically, T197V variants are more active than variants without that mutation, variants with T197L are inactive, here T197I decreased the activity of CA-cGAS-41, suggesting that valine is the largest hydrophobic residue allowed at this position. R158S adds a capping residue on the carbonyl end of the N-terminal segment of the spine helix but does not increase activity. Y200I instead of Y200F makes CA-cGAS-41 more like CA-cGAS-50, testing the interchangeability of the activating mutations. Mutations at position 207 are designed to destabilize the inactive conformation of the active site loop. S207G and S207V are as active as CA-cGAS-41, but S2071 is inactive. c, Recent work suggests phosphorylation at Y201 retains cGAS in the cytosol (32). However, in the active conformation this residue is well packed; phosphorylation would likely inhibit activation, but it is unclear how phosphorylation might affect CA-cGAS activity. To mimic phosphorylation at residue 201, we introduced the mutation Y201E. We also made Y201F or Y201W mutations to prevent phosphorylation at that site. Y201E completely knocks out activity in WT cGAS and significantly lowers it in some, but not all, CA-cGAS variants. Mutating Y201 to phenylalanine or tryptophan had little effect on cGAS activity. d, The activity of CA-cGAS-22, -41, -42, and -50 mutations in an hcGAS background.

### DETAILED DESCRIPTION

All references cited are herein incorporated by reference in their entirety. As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R). cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Any N-terminal methionines in the polypeptides of the disclosure are optional, and may be present or absent.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above.” and “below” and words of similar import, when used in this application, shall refer to this application as a whole and not to any particular portions of the application.

In one aspect, the disclosure provides polypeptides comprising an amino acid sequence at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:1 or 2,

SEQ ID NO: 1
(DAAP)GASKLRAVLEKLKLSRDDISTAAGMVKGVVDHLLLRLKCDSAFR
GVGLLNTGSYYEHVKISAPNEFDVMFKLEVPRIQLEEYSNTRAYYFVKFK
RNPKENPLSQFLEGEILSASKMLSKFRKIIKEEINDIKDTDVIMKRKRGG
SPAVTLLISEKISVDITLALESKSSWPASTQEGLRIQNWLSAKVRKQLRL
KPFYLVPKHAKEGNGFQEETWRLSFSHIEKEILNNHGKSKTCCENKEEKC
CRKDCLKLMKYLLEQLKERFKDKKHLDKFSSYHVKTAFFHVCTQNPQDSQ
WDRKDLGLCFDNCVTYFLQCLRTEKLENYFIPEFNLFSSNLIDKRSKEFL
TKQIEYERNNEFPVFDEF (WT Human cGas, truncated)
(SEQ ID NO: 2)
(M)QPWHGKAMQRASEAGATAPKASARNARGAPMDPTESPAAPEAALPKA
GKFGPARKSGSRQKKSAPDTQERPPVRATGARAKKAPQRAQDTQPSDATS
APGAEGLEPPAAREPALSRAGSCRQRGARCSTKPRPPPGPWDVPSPGLPV
SAPILVRRDAAPGASKLRAVLEKLKLSRDDISTAAGMVKGVVDHLLLRLK
CDSAFRGVGLLNTGSYYEHVKISAPNEFDVMFKLEVPRIQLEEYSNTRAY
YFVKFKRNPKENPLSQFLEGEILSASKMLSKFRKIIKEEINDIKDTDVIM
KRKRGGSPAVTLLISEKISVDITLALESKSSWPASTQEGLRIQNWLSAKV
RKQLRLKPFYLVPKHAKEGNGFQEETWRLSFSHIEKEILNNHGKSKTCCE
NKEEKCCRKDCLKLMKYLLEQLKERFKDKKHLDKFSSYHVKTAFFHVCTQ
NPQDSQWDRKDLGLCFDNCVTYFLWCLRTEKLENYFIPEFNLFSSNLIDK
RSKEFLTKQIEYERNNEFPVFDEF (Full length Human cGas)

wherein residues in parentheses are optional and may be present or absent;
wherein relative to residue numbering in SEQ ID NO:1:
residue 56 is G, residue 57 is S, residue 60 is E, residue 69 is E, and residue 71 is D; and
wherein one or both of the following are true:
(a) residue 19 is P and residue 20 is A; or residue 19 is D and residue 20 is P; and
(b) residue 54 is P, L, or F and residue 58 is F, I, W, or L.

The polypeptides disclosed herein are constitutively active Cyclic GMP-AMP Synthase (cGAS) mutants), as disclosed in detail in the examples that follow, and thus can be used, for example, as adjuvants for vaccine administration and to stimulate anti-tumor immunity.

As disclosed herein, the inventors developed a simple, knowledge-based two-state design protocol that can be generally applied to stabilize specific conformations of dynamic proteins where target and off-target structures are known. Extensive studies were carried out, as detailed in the examples below, to identify key residues involved in the structural rearrangements between DNA-free and DNA bound conformations of cGAS to arrive at the DNA-independent, constitutively active cGAS mutants of the disclosure, and to verify that other residues can vary quite broadly.

SEQ ID NO:1 is the amino acid sequence of truncated human cGAS, and SEQ ID NO:2 is the amino acid sequence of full length cGAS, which is 156 amino acids longer than SEQ ID NO:1. Residue numbering to define amino acid sequences is relative to SEQ ID NO:1 (the truncated version). Thus, if residue numbering is relative to the amino acid of SEQ ID NO:2, then 156 amino acids would be added. For example:
residue 56 relative to SEQ ID NO:1 is G, while residue 212 relative to SEQ ID NO:2 is G;
residue 57 relative to SEQ ID NO:1 is S, while residue 213 relative to SEQ ID NO:2 is S;
residue 60 relative to SEQ ID NO:1 is E, while residue 216 relative to SEQ ID NO:2 is E
residue 69 relative to SEQ ID NO:1 is E, while residue 225 relative to SEQ ID NO:2 is E; and
residue 71 relative to SEQ ID NO:1 is D, while residue 227 relative to SEQ ID NO:2 is D.

Those of skill in the art will be able, based on the disclosure, determine the amino acid residue relative to SEQ ID NO:2 for other specified positions relative to SEQ ID NO:1 disclosed herein.

In one embodiment, residue 19 is P and residue 20 is A relative to SEQ ID NO:1. In another embodiment, residue 19 is D and residue 20 is P relative to SEQ ID NO:1.

In various further embodiments, the following positions relative to SEQ ID NO:1 numbering may be one or more of the following:
(a) residue 54 is P, L, or F and residue 58 is F, I, W, or L;
(b) residue 55 is V, I, A, S, or G, preferably wherein residue 55 is V or I, most preferably wherein residue 55 is V;
(c) residue 61 is K;
(d) 1, 2, or all 3 of the following are true: residue 62 is V, residue 63 is K, residue 64 is I;
(e) one or more of the following is true: residue 17 is S; residue 18 is I; residue 59 is E, F, W, L, or I, preferably wherein residue 59 is F, W, L, or I, more preferably wherein residue 59 is F or W; residue 65 is G or V; residue 251 is M; and/or residue 255 is M.

In one embodiment, the polypeptide does not include the mutation or combination of mutations listed on a single line in Table 1, relative to SEQ ID NO: 1.

TABLE 1

K19D/R20P/T56I/Y59I/H62Q/V63I
K19D/R20P/Y59F/H62Q/V63I
K19D/R20P/Y59I/H62K/V63I
K19D/R20P/Y59F/H62K/V63I
K19D/R20P/Y59I/H62R/V63I
K19D/R20P/T56L/Y59F/V63F
K19D/R20P/Y59F/H62G/V63F
K19D/R20P/N55L/Y59F/H62Q/V63I
K19D/R20P/N55L/Y59I/H62K/V63I
K19D/R20P/N55R/Y59F/H62Q/V63I
K19D/R20P/N55R/Y59F/V63F
K19D/R20P/N55F/T56L/Y59F/H62R
K19D/R20P/K64L
K19D/R20P/N55W/I65V
L18I/K19D/R20P
L18V/K19D/R20P
R17P/K19D/R20P
R17P/L18V/K19D/R20P
R17P/K19D/R20P/I23Q/E25D
N55P/Y59F/H62Q/V63I
N55P/Y59V/H62K
N55P/T56I/Y59F/V63N
N55P/Y59F/V63F
T56V/Y59I/H62Q/V63F
T56I/Y59I/H62Q/V63I
Y59F/H62Q/V63I
Y59I/H62K/V63I
Y59F/H62K/V63I
T56L/Y59F/V62F
T56I/Y59F/V63I
N55L/Y59F/H62Q/V63I
N55L/Y59I/H62K/V63I
N55L/T56V/Y59F/H62K
N55L/Y59F/H62K
N55R/T56I/Y59I/H62K/V63I
N55F/T56L/Y59F/H62G/V63N
N55F/T56V/Y59I/H62K
N55F/T56L/Y59F/H62R
N55F/Y59F/V63I
K64L
R17P
L18P
K19P
R20P
N55W
N55F
Y59I
Y59F
L18P/R20P
L18P/K19D/R20P
K19D/R20P
R17P/L18I/K19D/R20P
R17P/L18I/K19P/R20P
R17P/L18V/K19D/R20P
R18P/K19D/R20P/I23Q
R18P/K19D/R20P/S24D
R18P/K19D/R20P/I23Q/E25D
N55P/Y59F/H62Q/V63I
N55P/Y59V/H62K
N55P/T56I/Y59F/V63N
N55P/Y59F/V63F
T56V/Y59I/H62Q/V63F
T56I/Y59I/H62Q/V63I
Y59F/H62Q/V63I
Y59I/H62K/V63I
Y59F/H62K/V63I
T56L/Y59F/V63F
T56I/Y59F/V63I
N56L/Y59F/H62Q/V63I
N56L/Y59I/H62K/V63I
N56L/T56V/Y59F/H62K
N56L/Y59F/H62K
N56R/T56I/Y59I/H62K
N56F/T56L/Y59F/H62G/V63N
N56F/T56V/Y59I/H62K
N56F/T56L/Y59F/H62R
N56F/Y59F/V63I
N69R/V73F
R17P
L18P
K19P
R20P
N55W
N55F
Y59I

In another embodiment, the polypeptide does not include the mutation or combination of mutations listed on a single line Table 2, relative to SEQ ID NO: 2.

TABLE 2

K175D/R176P/T212I/Y215I/H218Q/V219I
K175D/R176P/Y215F/H218Q/V219I
K175D/R176P/Y215I/H218K/V219I
K175D/R176P/Y215F/H218K/V219I
K175D/R176P/Y215I/H218R/V219I
K175D/R176P/T212L/Y215F/V219F
K175D/R176P/Y215F/H218G/V219F
K175D/R176P/N211L/Y215F/H218Q/V219I
K175D/R176P/N211L/Y215I/H218K/V219I
K175D/R176P/N211R/Y215F/H218Q/V219I
K175D/R176P/N211R/Y215F/V219F
K175D/R176P/N211F/T212L/Y215F/H218R
K175D/R176P/K220L
K175D/R176P/N211W/I221V
L174I/K175D/R176P
L174V/K175D/R176P
R173P/K175D/R176P
R173P/L174V/K175D/R176P
R173P/K175D/R176P/I179Q/E181D
N211P/Y215F/H218Q/V219I
N211P/Y215V/H218K
N211P/T212I/Y215F/V219N
N211P/Y215F/V219F
T212V/Y215I/H218Q/V219F
T212I/Y215I/H218Q/V219I
Y215F/H218Q/V219I
Y215I/H218K/V219I
Y215F/H218K/V219I
T212L/Y215F/V218F
T212I/Y215F/V219I
N211L/Y215F/H218Q/V219I
N211L/Y215I/H218K/V219I
N211L/T212V/Y215F/H218K
N211L/Y215F/H218K
N211R/T212I/Y215I/H218K/V219I
N211F/T212L/Y215F/H218G/V219N
N211F/T212V/Y215I/H218K
N211F/T212L/Y215F/H218R
N211F/Y215F/V219I
K220L
R173P
L174P
K175P
R176P
N211W
N211F
Y215I
Y215F
L174P/R176P
L174P/K175D/R176P
K175D/R176P
R173P/L174I/K175D/R176P
R173P/L174I/K175P/R176P
R173P/L174V/K175D/R176P
R174P/K175D/R176P/1179Q
R174P/K175D/R176P/S180D
R174P/K175D/R176P/I179Q/E181D
N211P/Y215F/H218Q/V219I
N211P/Y215V/H218K
N211P/T212I/Y215F/V219N
N211P/Y215F/V219F
T212V/Y215I/H218Q/V219F
T212I/Y215I/H218Q/V219I
Y215F/H218Q/V219I
Y215I/H218K/V219I
Y215F/H218K/V219I
T212L/Y215F/V219F
T212I/Y215F/V219I
N212L/Y215F/H218Q/V219I
N212L/Y215I/H218K/V219I
N212L/T212V/Y215F/H218K
N212L/Y215F/H218K
N212R/T212I/Y215I/H218K
N212F/T212L/Y215F/H218G/V219N
N212F/T212V/Y215I/H218K
N212F/T212L/Y215F/H218R
N212F/Y215F/V219I
N225R/V229F
R173P
L174P
K175P
R176P
N211W
N211F
Y215I

In another aspect, the disclosure provides polypeptides comprising an amino acid sequence at least 20%, 25%, 30%, 35% 40%, 45%, 50%, 55%, 60%, 65%, 70%, 75%, 80%, 85%, 90%, 91%, 92%, 93%, 94%, 95%, 96%, 97%, 98%, or 99% identical to the amino acid sequence of SEQ ID NO:3 or 4,

(SEQ ID NO: 3)
(SPD)KLKKVLDKLRLKRKDISEAAETVNKVVERLLRRMQKRESEEKGVE
QLNTGSYYEHVKISAPNEFDVMFKLEVPRIELQEYYETGAFYLVKFKRIP
RGNPLSHFLEGEVLSATKMLSKFRKIIKEEVKEIKDIDVSVEKEKPGSPA
VTLLIRNPEEISVDIILALESKGSWPISTKEGLPIQGWLGTKVRTNLRRE
PFYLVPKNAKDGNSFQGETWRLSFSHTEKYILNNHGIEKTCCESSGAKCC
RKECLKIMKYLLEQLKKEFQELDAFCSYHVKTAIFHMWTQDPQDSQWDPR
NLSSCFDKLLAFFLECLRTEKLDHYFIPKFNLFSQELIDRKSKEFLSKKI
EYERNNGFPIFDKL WT mouse cGAS, truncated
(SEQ ID NO: 4)
(M)EDPRRRTTAPRAKKPSAKRAPTQPSRTRAHAESCGPQRGARSRRAER
DGDTTEKPRAPGPRVHPARATELTKDAQPSAMDAAGATARPAVRVPQQQA
ILDPELPAVREPQPPADPEARKVVRGPSHRRGARSTGQPRAPRGSRKEPD
KLKKVLDKLRLKRKDISEAAETVNKVVERLLRRMQKRESEFKGVEQLNTG
SYYEHVKISAPNEFDVMFKLEVPRIELQEYYETGAFYLVKFKRIPRGNPL
SHFLEGEVLSATKMLSKFRKIIKEEVKEIKDIDVSVEKEKPGSPAVTLLI
RNPEEISVDIILALESKGSWPISTKEGLPIQGWLGTKVRTNLRREPFYLV
PKNAKDGNSFQGETWRLSFSHTEKYILNNHGIEKTCCESSGAKCCRKECL
KMKYLLEQLKKEFQELDAFCSYHVKTAIFHMWTQDPQDSQWDPRNLSSCF
DKLLAFFLECLRTEKLDHYFIPKENLFSQELIDRKSKEFLSKKIEYERNN
GFPIFDKL Full length mouse cGAS

wherein residues in parentheses are optional and may be present or absent;
wherein relative to residue numbering in SEQ ID NO:3:
residue 53 is G, residue 54 is S, residue 57 is E, residue 66 is E, and residue 68 is D; and
wherein one or both of the following are true:
(a) residue 15 is P and residue 16 is A; or residue 15 is D and residue 16 is P; and
(b) residue 51 is P, L, or F and residue 55 is F, I, W, or L.

SEQ ID NO:3 is the amino acid sequence of truncated mouse cGAS, and SEQ ID NO:4 is the amino acid sequence of full length mouse cGAS, which is 145 amino acids longer than SEQ ID NO:4. Residue numbering to define amino acid sequences is relative to SEQ ID NO:3 (the truncated version). Thus, if residue numbering is relative to the amino acid of SEQ ID NO:4, then 145 amino acids would be added.

In one embodiment, residue 15 is P and residue 16 is A relative to SEQ ID NO: 3. In another embodiment, wherein residue 15 is D and residue 16 is P relative to SEQ ID NO: 3.

In various further embodiments, the following positions relative to SEQ ID NO:3 numbering may be one or more of the following:
(a) residue 51 is P, L, or F and residue 55 is F, I, W, or L;
(b) residue 52 is V, I, A, G, or S; preferably wherein residue 52 is V or I, or more preferably wherein residue 52 is V;
(c) residue 58 is K;
(d) 1, 2, or all 3 are true: residue 59 is V, residue 60 is K, residue 61 is I;
(e) one or more of the following is true: residue 13 is S; residue 14 is I; residue 56 is E, F, W, L, or I, preferably wherein residue 56 is F, W, L, or I, more preferably wherein residue 56 is F ow W; residue 62 is G or V; residue 250 is M; and/or residue 254 is M.

In another embodiment, the polypeptide does not include the mutation or combination of mutations listed on a single line Table 3, relative to SEQ ID NO: 3.

TABLE 3

K15D/R16P/T52I/Y55I/H58Q/V59I
K15D/R16P/Y55F/H58Q/V59I
K15D/R16P/Y55I/H58K/V59I
K15D/R16P/Y55F/H58K/V59I
K15D/R16P/Y55I/H58R/V59I
K15D/R16P/T52L/Y55F/V59F
K15D/R16P/Y55F/H58G/V59F
K15D/R16P/N51L/Y55F/H58Q/V59I
K15D/R16P/N51L/Y55I/H58K/V59I
K15D/R16P/N51R/Y55F/H58Q/V59I
K15D/R16P/N51R/Y55F/V59F
K15D/R16P/N51F/T52L/Y55F/H58R
K15D/R16P/K60L
K15D/R16P/N51W/I61V
L14I/K15D/R16P
L14V/K15D/R16P
R13P/K15D/R16P
R13P/L14V/K15D/R16P
R13P/K15D/R16P/I19Q/E21D
N51P/Y55F/H58Q/V59I
N51P/Y55V/H58K
N51P/T52I/Y55F/V59N
N51P/Y55F/V59F
T52V/Y55I/H58Q/V59F
T52I/Y55I/H58Q/V59I
Y55F/H58Q/V59I
Y55I/H58K/V59I
Y55F/H58K/V59I
T52L/Y55F/V58F
T52I/Y55F/V59I
N51L/Y55F/H58Q/V59I
N51L/Y55I/H58K/V59I
N51L/T52V/Y55F/H58K
N51L/Y55F/H58K
N51R/T52I/Y55I/H58K/V59I
N51F/T52L/Y55F/H58G/V59N
N51F/T52V/Y55I/H58K
N51F/T52L/Y55F/H58R
N51F/Y55F/V59I
K60L
R13P
L14P
K15P
R16P
N51W
N51F
Y55I
Y55F
L14P/R16P
L14P/K15D/R16P
K15D/R16P
R13P/L14I/K15D/R16P
R13P/L14I/K15P/R16P
R13P/L14V/K15D/R16P
R14P/K15D/R16P/I19Q
R14P/K15D/R16P/S20D
R14P/K15D/R16P/I19Q/E21D
N51P/Y55F/H58Q/V59I
N51P/Y55V/H58K
N51P/T52I/Y55F/V59N
N51P/Y55F/V59F
T52V/Y55I/H58Q/V59F
T52I/Y55I/H58Q/V59I
Y55F/H58Q/V59I
Y55I/H58K/V59I
Y55F/H58K/V59I
T52L/Y55F/V59F
T52I/Y55F/V59I
N52L/Y55F/H58Q/V59I
N52L/Y55I/H58K/V59I
N52L/T52V/Y55F/H58K
N52L/Y55F/H58K
N52R/T52I/Y55I/H58K
N52F/T52L/Y55F/H58G/V59N
N52F/T52V/Y55I/H58K
N52F/T52L/Y55F/H58R
N52F/Y55F/V59I
N65R/V69F
R13P
L14P
K15P
R16P
N51W
N51F
Y55I

In another embodiment, the polypeptide does not include the mutation or combination of mutations listed on a single line Table 4, relative to SEQ ID NO: 4.

TABLE 4

K160P/N196L/T197V/Y200F
K160D/R161P/T197I/Y200I/H203Q/V204I
K160D/R161P/Y200F/H203Q/V204I
K160D/R161P/Y200I/H203K/V204I
K160D/R161P/Y200F/H203K/V204I
K160D/R161P/Y200I/H203R/V204I
K160D/R161P/T197L/Y200F/V204F
K160D/R161P/Y200F/H203G/V204F
K160D/R161P/N196L/Y200F/H203Q/V204I
K160D/R161P/N196L/Y200I/H203K/V204I
K160D/R161P/N196R/Y200F/H203Q/V204I
K160D/R161P/N196R/Y200F/V204F
K160D/R161P/N196F/T197L/Y200F/H203R
K160D/R161P/K205L/
K160D/R161P/N196W/I206V
L159I/K160D/R161P
L159V/K160D/R161P
R158P/K160D/R161P
R158P/L159V/K160D/R161P
R158P/K160D/R161P/I164Q/E166D
N196P/Y200F/H203Q/V204I
N196P/Y200V/H203K
N196P/T197I/Y200F/V204N
N196P/Y200F/V204F
T197V/Y200I/H203Q/V204F
T197I/Y200I/H203Q/V204I
Y200F/H203Q/V204I
Y200I/H203K/V204I
Y200F/H203K/V204I
T197L/Y2003/V203F
T197I/Y2003/V204I
N196L/Y2003/H203Q/V204I
N196L/Y200I/H203K/V204I
N196L/T197V/Y200F/H203K
N196L/Y2003/H203K
N196R/T197I/Y200I/H203K/V204I
N196F/T197L/Y200F/H203G/V204N
N196F/T197V/Y200I/H203K
N196F/T197L/Y200F/H203R
N196F/Y200F/V204I
K205L
R158P
L159P
K160P
R161P
N196W
N196F
Y200I
Y200F
L159P/R161P
L159P/K160D/R161P
K160D/R161P
R158P/L159IP/K160D/R161P
R158P/L159I/K160P/R161P
R158P/L159V/K160D/R161P
R159P/K160D/R161P/I164Q
R159P/K160D/R161P/S165D
R159P/K160D/R161P/I164Q/E166D
N196P/Y200F/H203Q/V204I
N196P/Y200V/H203K
N196P/T197I/Y200F/V204N
N196P/Y200F/V204F
T197V/Y200I/H203Q/V204F
T197I/Y200I/H203Q/V204I
Y200F/H203Q/V204I
Y200I/H203K/V204I
Y200F/H203K/V204I
T197L/Y200F/V204F
T197I/Y2003/V204I
N197L/Y200F/H203Q/V204I
N197L/Y200I/H203K/V204I
N197L/T197V/Y200F/H203K
N197L/Y200F/H203K
N197R/T197I/Y200I/H203K
N197F/T197L/Y200F/H203G/V204N
N197F/T197V/Y200I/H203K
N197F/T197L/Y200F/H203R
N197F/Y200F/V204I
N210R/V2143
R158P
L159P
K160P
R161P
N196W
N196F
Y200I

In another embodiment, the polypeptides comprise an amino acid sequence at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:5-59, wherein any N-terminal methionines are optional and may be present or absent.

| K395M/ | DAAPGASKLRAVLEKLKLSRDDISTAAGMVKGVVDHLLLRLKCDSAFRGVGLLNTGSYYEHVKISAPNEF | |
|---|---|---|
| K399M | DVMFKLEVPRIQLEEYSNTRAYYFVKFKRNPKENPLSQFLEGEILSASKMLSKFRKIIKEEINDIKDTDV | |
| | IMKRKRGGSPAVTLLISEKISVDITLALESKSSWPASTQEGLRIQNWLSAKVRKQLRLKPFYLVPKHAKE | |
| | GNGFQEETWRLSFSHIEKEILNNHGKSKTCCENKEEKCCRMDCLMLMKYLLEQLKERFKDKKHLDKFSSY | |
| | HVKTAFFHVCTQNPQDSQWDRKDLGLCFDNCVTYFLQCLRTEKLENYFIPEFNLFSSNLIDKRSKEFLTK | |
| | QIEYERNNEFPVFDEF (SEQ ID NO: 5) | |
| K395M/ | MQPWHGKAMQRASEAGATAPKASARNARGAPMDPTESPAAPEAALPKAGKFGPARKSGSRQKKSAPDTQE | |
| K399M | RPPVRATGARAKKAPQRAQDTQPSDATSAPGAEGLEPPAAREPALSRAGSCRQRGARCSTKPRPPPGPWD | |
| FL | VPSPGLPVSAPILVRRDAAPGASKLRAVLEKLKLSRDDISTAAGMVKGVVDHLLLRLKCDSAFRGVGLLN | |
| | TGSYYEHVKISAPNEFDVMFKLEVPRIQLEEYSNTRAYYFVKFKRNPKENPLSQFLEGEILSASKMLSKF | |
| | RKIIKEEINDIKDTDVIMKRKRGGSPAVTLLISEKISVDITLALESKSSWPASTQEGLRIQNWLSAKVRK | |
| | QLRLKPFYLVPKHAKEGNGFQEETWRLSFSHIEKEILNNHGKSKTCCENKEEKCCRMDCLMLMKYLLEQL | |
| | KERFKDKKHLDKFSSYHVKTAFFHVCTQNPQDSQWDRKDLGLCFDNCVTYFLQCLRTEKLENYFIPEENL | |
| | FSSNLIDKRSKEFLTKQIEYERNNEFPVEDEF (SEQ ID NO: 6) | |
| K395M/ | SPDKLKKVLDKLRLKRKDISEAAETVNKVVERLLRRMQKRESEFKGVEQLNTGSYYEHVKISAPNEFDVM | |
| K399M | FKLEVPRIELQEYYETGAFYLVKFKRIPRGNPLSHFLEGEVLSATKMLSKFRKIIKEEVKEIKDIDVSVE | |
| mcGAS | KEKPGSPAVTLLIRNPEEISVDIILALESKGSWPISTKEGLPIQGWLGTKVRTNLRREPFYLVPKNAKDG | |
| | NSFQGETWRLSFSHTEKYILNNHGIEKTCCESSGAKCCRMECLMLMKYLLEQLKKEFQELDAFCSYHVKT | |
| | AIFHMWTQDPQDSQWDPRNLSSCFDKLLAFFLECLRTEKLDHYFIPKFNLFSQELIDRKSKEFLSKKIEY | |
| | ERNNGFPIFDKL (SEQ ID NO: 7) | |
| K395M/ | MEDPRRRTTAPRAKKPSAKRAPTQPSRTRAHAESCGPQRGARSRRAERDGDTTEKPRAPGPRVHPARATE | |
| K399M | LTKDAQPSAMDAAGATARPAVRVPQQQAILDPELPAVREPQPPADPEARKVVRGPSHRRGARSTGQPRAP | |
| FL | RGSRKEPDKLKKVLDKLRLKRKDISEAAETVNKVVERSLRRMQKRESEFKGVEQLNTGSYYEHVKISAPN | |
| mCGAS | EFDVMFKLEVPRIELQEYYETGAFYLVKFKRIPRGNPLSHFLEGEVLSATKMLSKFRKIIKEEVKEIKDI | |
| | DVSVEKEKPGSPAVTLLIRNPEEISVDIILALESKGSWPISTKEGLPIQGWLGTKVRTNLRREPFYLVPK | |
| | NAKDGNSFQGETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQELIDRKSKEFLS | |
| | KKIEYERNNGFPIFDKL (SEQ ID NO: 8) | |
| CA- | DAAPGASKLRAVLEKLKLDPDDISTAAGMVKGVVDHLLLRLKCDSAFRGVGLLPVGSFYE | K160D/ |
| cGAS- | KVKISAPNEFDVMFKLEVPRIQLEEYSNTRAYYFVKFKRNPKENPLSQFLEGEILSASKM | R161P/ |
| 22 | LSKFRKIIKEEINDIKDTDVIMKRKRGGSPAVTLLISEKISVDITLALESKSSWPASTQE | N196P/ |
| | GLRIQNWLSAKVRKQLRLKPFYLVPKHAKEGNGFQEETWRLSFSHIEKEILNNHGKSKTC | T197V/ |
| | CENKEEKCCRKDCLKLMKYLLEQLKERFKDKKHLDKFSSYHVKTAFFHVCTQNPQDSQWD | Y200F/ |
| | RKDLGLCFDNCVTYFLQCLRTEKLENYFIPEFNLFSSNLIDKRSKEFLTKQIEYERNNEF | H203K |
| | PVFDEF (SEQ ID NO: 9) | |
| CA- | DAAPGASKLRAVLEKLKLDPDDISTAAGMVKGVVDHLLLRLKCDSAFRGVGLLLVGSFYE | K160D/ |
| cGAS- | KVKISAPNEFDVMFKLEVPRIQLEEYSNTRAYYFVKFKRNPKENPLSQFLEGEILSASKM | R161P/ |
| 41 | LSKFRKIIKEEINDIKDTDVIMKRKRGGSPAVTLLISEKISVDITLALESKSSWPASTQE | N196L/ |
| | GLRIQNWLSAKVRKQLRLKPFYLVPKHAKEGNGFQEETWRLSFSHIEKEILNNHGKSKTC | T197V/ |
| | CENKEEKCCRKDCLKLMKYLLEQLKERFKDKKHLDKFSSYHVKTAFFHVCTQNPQDSQWD | Y200F/ |
| | RKDLGLCFDNCVTYFLQCLRTEKLENYFIPEFNLESSNLIDKRSKEFLTKQIEYERNNEF | H203K |
| | PVFDEF (SEQ ID NO: 10) | |
| CA- | DAAPGASKLRAVLEKLKLDPDDISTAAGMVKGVVDHLLLRLKCDSAFRGVGLLLTGSFYE | K160D/ |
| cGAS- | KVKISAPNEFDVMFKLEVPRIQLEEYSNTRAYYFVKFKRNPKENPLSQFLEGEILSASKM | R161P/ |
| 42 | LSKFRKIIKEEINDIKDTDVIMKRKRGGSPAVTLLISEKISVDITLALESKSSWPASTQE | N196L/ |
| | GLRIQNWLSAKVRKQLRLKPFYLVPKHAKEGNGFQEETWRLSFSHIEKEILNNHGKSKTC | T197V/ |
| | CENKEEKCCRKDCLKLMKYLLEQLKERFKDKKHLDKFSSYHVKTAFFHVCTQNPQDSQWD | Y200F/ |
| | RKDLGLCFDNCVTYFLQCLRTEKLENYFIPEENLFSSNLIDKRSKEFLTKQIEYERNNEF | H203K |
| | PVFDEF (SEQ ID NO: 11) | |
| CA- | DAAPGASKLRAVLEKLKLDPDDISTAAGMVKGVVDHLLLRLKCDSAFRGVGLLFVGSIYE | K160D/ |
| cGAS- | KVKISAPNEFDVMFKLEVPRIQLEEYSNTRAYYFVKFKRNPKENPLSQFLEGEILSASKM | R161P/ |
| 50 | LSKFRKIIKEEINDIKDTDVIMKRKRGGSPAVTLLISEKISVDITLALESKSSWPASTQE | FN196/ |
| | GLRIQNWLSAKVRKQLRLKPFYLVPKHAKEGNGFQEETWRLSFSHIEKEILNNHGKSKTC | T197V/ |
| | CENKEEKCCRKDCLKLMKYLLEQLKERFKDKKHLDKFSSYHVKTAFFHVCTQNPQDSQWD | Y200I/ |
| | RKDLGLCFDNCVTYFLQCLRTEKLENYFIPEFNLFSSNLIDKRSKEFLTKQIEYERNNEF | H203K |
| | PVFDEF (SEQ ID NO: 12) | |
| Full | MQPWHGKAMQRASEAGATAPKASARNARGAPMDPTESPAAPEAALPKAGKFGPARKSGSR | K160D/ |
| Length | QKKSAPDTQERPPVRATGARAKKAPQRAQDTQPSDATSAPGAEGLEPPAAREPALSRAGS | R161P/ |
| CA- | CRQRGARCSTKPRPPPGPWDVPSPGLPVSAPILVRRDAAPGASKLRAVLEKLKLDPDDIS | N196P/ |
| cGAS- | TAAGMVKGVVDHLLLRLKCDSAFRGVGLLPVGSFYEKVKISAPNEFDVMFKLEVPRIQLE | T197V/ |
| 22 | EYSNTRAYYFVKFKRNPKENPLSQFLEGEILSASKMLSKFRKIIKEEINDIKDTDVIMKR | Y200F/ |
| | KRGGSPAVTLLISEKISVDITLALESKSSWPASTQEGERLQNWLSAKVRKQLRLKPFYLV | H203K |
| | PKHAKEGNGFQEETWRLSFSHIEKEILNNHGKSKTCCENKEEKCCRKDCLKLMKYLLEQL | |
| | KERFKDKKHLDKFSSYHVKTAFFHVCTQNPQDSQWDRKDLGLCFDNCVTYFLQCLRTEKL | |
| | ENYFIPEFNLFSSNLIDKRSKEFLTKQIEYERNNEFPVFDEF (SEQ ID NO: 13) | |
| Full | MQPWHGKAMQRASEAGATAPKASARNARGAPMDPTESPAAPEAALPKAGKFGPARKSGSR | K160D/ |
| Length | QKKSAPDTQERPPVRATGARAKKAPQRAQDTQPSDATSAPGAEGLEPPAAREPALSRAGS | R161P/ |
| CA- | CRQRGARCSTKPRPPPGPWDVPSPGLPVSAPILVRRDAAPGASKLRAVLEKLKLDPDDIS | N196L/ |
| cGAS- | TAAGMVKGVVDHLLLRLKCDSAFRGVGLLLVGSFYEKVKISAPNEFDVMFKLEVPRIQLE | T197V/ |
| 41 | EYSNTRAYYFVKFKRNPKENPLSQFLEGEILSASKMLSKFRKIIKEEINDIKDTDVIMKR | Y200F/ |
| | KRGGSPAVTLLISEKISVDITLALESKSSWPASTQEGGRIQNWLSAKVRKQLRLKPFYLV | H203K |
| | PKHAKEGNGFQEETWRLSFSHIEKEILNNHGKSKTCCENKEEKCCRKDCLKLMKYLLEQL | |
| | KERFKDKKHLDKFSSYHVKTAFFHVCTQNPQDSQWDRKDLGLCFDNCVTYFLQCLRTEKL | |
| | ENYFIPEFNLFSSNLIDKRSKEFLTKQIEYERNNEFPVFDEF (SEQ ID NO: 14) | |
| Full | MQPWHGKAMQRASEAGATAPKASARNARGAPMDPTESPAAPEAALPKAGKFGPARKSGSR | K160D/ |
| Length | QKKSAPDTQERPPVRATGARAKKAPQRAQDTQPSDATSAPGAEGLEPPAAREPALSRAGS | R161P/ |
| CA- | CRQRGARCSTKPRPPPGPWDVPSPGLPVSAPILVRRDAAPGASKLRAVLEKLKLDPDDIS | N196L/ |
| cGAS- | TAAGMVKGVVDHLLLRLKCDSAFRGVGLLLTGSFYEKVKISAPNEFDVMFKLEVPRIQLE | Y200F/ |
| 42 | EYSNTRAYYFVKFKRNPKENPLSQFLEGEILSASKMLSKFRKIIKEEINDIKDTDVIMKR | H203K |
| | KRGGSPAVTLLISEKISVDITLALESKSSWPASTQEGERLQNWLSAKVRKQLRLKPFYLV | |
| | PKHAKEGNGFQEETWRLSFSHIEKEILNNHGKSKTCCENKEEKCCRKDCLKLMKYLLEQL | |
| | KERFKDKKHLDKFSSYHVKTAFFHVCTQNPQDSQWDRKDLGLCFDNCVTYFLQCLRTEKL | |
| | ENYFIPEFNLFSSNLIDKRSKEFLTKQIEYERNNEFPVFDEF (SEQ ID NO: 15) | |
| Full | MQPWHGKAMQRASEAGATAPKASARNARGAPMDPTESPAAPEAALPKAGKFGPARKSGSR | K160D/ |
| Length | QKKSAPDTQERPPVRATGARAKKAPQRAQDTQPSDATSAPGAEGLEPPAAREPALSRAGS | R161P/ |
| CA- | CRQRGARCSTKPRPPPGPWDVPSPGLPVSAPILVRRDAAPGASKLRAVLEKLKLDPDDIS | N196F/ |
| cGAS- | TAAGMVKGVVDHLLLRLKCDSAFRGVGLLFVGSIYEKVKISAPNEFDVMFKLEVPRIQLE | T197V/ |
| 50 | EYSNTRAYYFVKFKRNPKENPLSQFLEGEILSASKMLSKFRKIIKEEINDIKDTDVIMKR | Y200I/ |
| | KRGGSPAVTLLISEKISVDITLALESKSSWPASTQEGLRIQNWLSAKVRKQLRLKPFYLV | H203K |
| | PKHAKEGNGFQEETWRLSFSHIEKEILNNHGKSKTCCENKEEKCCRKDCLKLMKYLLEQL | |
| | KERFKDKKHLDKFSSYHVKTAFFHVCTQNPQDSQWDRKDLGLCFDNCVTYFLQCLRTEKL | |
| | ENYFIPEFNLFSSNLIDKRSKEFLTKQIEYERNNEFPVFDEF (SEQ ID NO: 16) | |
| CA- | SPDKLKKVLDKLRLDPKDISEAAETVNKVVERLLRRMQKRESEFKGVEQLPVGSFYEKVK | K160D/ |
| mcGAS- | ISAPNEFDVMFKLEVPRIELQEYYETGAFYLVKFKRIPRGNPLSHFLEGEVLSATKMLSK | R161P/ |
| 22 | FRKIIKEEVKEIKDIDVSVEKEKPGSPAVTLLIRNPEEISVDIILALESKGSWPISTKEG | N196P/ |
| | LPIQGWLGTKVRTNLRREPFYLVPKNAKDGNSFQGETWRLSFSHTEKYILNNHGIEKTCC | T197V/ |
| | ESSGAKCCRMECLMLMKYLLEQLKKEFQELDAFCSYHVKTAIFHMWTQDPQDSQWDPRNL | Y200F/ |
| | SSCFDKLLAFFLECLRTEKLDHYFIPKFNLFSQELIDRKSKEFLSKKIEYERNNGFPIFD | H203K |
| | KL (SEQ ID NO: 17) | |
| CA- | SPDKLKKVLDKLRLDPKDISEAAETVNKVVERLLRRMQKRESEFKGVEQLLVGSFYEKVK | K160D/ |
| mcGAS- | ISAPNEFDVMFKLEVPRIELQEYYETGAFYLVKFKRIPRGNPLSHFLEGEVLSATKMLSK | R161P/ |
| 41 | FRKIIKEEVKEIKDIDVSVEKEKPGSPAVTLLIRNPEEISVDIILALESKGSWPISTKEG | N196L/ |
| | LPIQGWLGTKVRTNLRREPFYLVPKNAKDGNSFQGETWRLSFSHTEKYILNNHGIEKTCC | T197V/ |
| | ESSGAKCCRMECLMLMKYLLEQLKKEFQELDAFCSYHVKTAIFHMWTQDPQDSQWDPRNL | Y200F/ |
| | SSCFDKLLAFFLECLRTEKLDHYFIPKFNLFSQELIDRKSKEFLSKKIEYERNNGFPIFD | H203K |
| | KL (SEQ ID NO: 18) | |
| CA- | SPDKLKKVLDKLRLDPKDISEAAETVNKVVERLLRRMQKRESEFKGVEQLLTGSFYEKVK | K160D/ |
| mcGAS- | ISAPNEFDVMFKLEVPRIELQEYYETGAFYLVKFKRIPRGNPLSHFLEGEVLSATKMLSK | R161P/ |
| 42 | FRKIIKEEVKEIKDIDVSVEKEKPGSPAVTLLIRNPEEISVDIILALESKGSWPISTKEG | N196L/ |
| | LPIQGWLGTKVRTNLRREPFYLVPKNAKDGNSFQGETWRLSFSHTEKYILNNHGIEKTCC | Y200F/ |
| | ESSGAKCCRMECLMLMKYLLEQLKKEFQELDAFCSYHVKTAIFHMWTQDPQDSQWDPRNL | H203K |
| | SSCFDKLLAFFLECLRTEKLDHYFIPKFNLFSQELIDRKSKEFLSKKIEYERNNGFPIFD | |
| | KL (SEQ ID NO: 19) | |
| CA- | SPDKLKKVLDKLRLDPKDISEAAETVNKVVERLLRRMQKRESEFKGVEQLFVGSIYEKVK | K160D/ |
| mcGAS- | ISAPNEFDVMFKLEVPRIELQEYYETGAFYLVKFKRIPRGNPLSHFLEGEVLSATKMLSK | R161P/ |
| 50 | FRKIIKEEVKEIKDIDVSVEKEKPGSPAVTLLIRNPEEISVDIILALESKGSWPISTKEG | N196F/ |
| | LPIQGWLGTKVRTNLRREPFYLVPKNAKDGNSFQGETWRLSFSHTEKYILNNHGIEKTCC | T197V/ |
| | ESSGAKCCRMECLMLMKYLLEQLKKEFQELDAFCSYHVKTAIFHMWTQDPQDSQWDPRNL | Y200I/ |
| | SSCFDKLLAFFLECLRTEKLDHYFIPKFNLFSQELIDRKSKEFLSKKIEYERNN (SEQ | H203K |
| | ID NO: 20) | |
| Full | MEDPRRRTTAPRAKKPSAKRAPTQPSRTRAHAESCGPQRGARSRRAERDGDTTEKPRAPG | K160D/ |
| Length | PRVHPARATELTKDAQPSAMDAAGATARPAVRVPQQQAILDPELPAVREPQPPADPEARK | R161P |
| CA- | VVRGPSHRRGARSTGQPRAPRGSRKEPDKLKKVLDKLRLDPKDISEAAETVNKVVERLLR | |
| mcGAS- | RMQKRESEFKGVEQLNTGSYYEHVKISAPNEFDVMFKLEVPRIELQEYYETGAFYLVKFK | |
| 04 | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 21) | |
| Full | MEDPRRRTTAPRAKKPSAKRAPTQPSRTRAHAESCGPQRGARSRRAERDGDTTEKPRAPG | K160D/ |
| Length | PRVHPARATELTKDAQPSAMDAAGATARPAVRVPQQQAILDPELPAVREPQPPADPEARK | R161P/ |
| CA- | VVRGPSHRRGARSTGQPRAPRGSRKEPDKLKKVLDKLRLDPKDISEAAETVNKVVERLLR | N196P/ |
| mcGAS- | RMQKRESEFKGVEQLPVGSFYEKVKISAPNEFDVMFKLEVPRIELQEYYETGAFYLVKFK | T197V/ |
| 22 | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 22) | |
| Full | MEDPRRRTTAPRAKKPSAKRAPTQPSRTRAHAESCGPQRGARSRRAERDGDTTEKPRAPG | K160D/ |
| Length | PRVHPARATELTKDAQPSAMDAAGATARPAVRVPQQQAILDPELPAVREPQPPADPEARK | R161P/ |
| CA- | VVRGPSHRRGARSTGQPRAPRGSRKEPDKLKKVLDKLRLDPKDISEAAETVNKVVERLLR | N196L/ |
| mcGAS- | RMQKRESEFKGVEQLLVGSFYEKVKISAPNEFDVMFKLEVPRIELQEYYETGAFYLVKFK | T197V/ |
| 41 | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 23) | |
| Full | MEDPRRRTTAPRAKKPSAKRAPTQPSRTRAHAESCGPQRGARSRRAERDGDTTEKPRAPG | K160D/ |
| Length | PRVHPARATELTKDAQPSAMDAAGATARPAVRVPQQQAILDPELPAVREPQPPADPEARK | R161P/ |
| CA- | VVRGPSHRRGARSTGQPRAPRGSRKEPDKLKKVLDKLRLDPKDISEAAETVNKVVERLLR | N196L/ |
| mcGAS- | RMQKRESEFKGVEQLLTGSFYEKVKISAPNEFDVMFKLEVPRIELQEYYETGAFYLVKFK | Y200F/ |
| 42 | RIPRGNPLSHFLEGEVLSATKMLSKFRKIIKEEVKEIKDIDVSVEKEKPGSPAVTLLIRN | H203K |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 24) | |
| Full | MEDPRRRTTAPRAKKPSAKRAPTQPSRTRAHAESCGPQRGARSRRAERDGDTTEKPRAPG | K160D/ |
| Length | PRVHPARATELTKDAQPSAMDAAGATARPAVRVPQQQAILDPELPAVREPQPPADPEARK | R161P/ |
| CA- | VVRGPSHRRGARSTGQPRAPRGSRKEPDKLKKVLDKLRLDPKDISEAAETVNKVVERLLR | N196F/ |
| mcGAS- | RMQKRESEFKGVEQLFVGSIYEKVKISAPNEFDVMFKLEVPRIELQEYYETGAFYLVKFK | T197V/ |
| 50 | RIPRGNPLSHFLEGEVLSATKMLSKFRKIIKEEVKEIKDIDVSVEKEKPGSPAVTLLIRN | Y200I/ |
| | PEEISVDIILALESKGSWPISTKEGLPIQGWLGTKVRTNLRREPFYLVPKNAKDGNSFQG | H203K |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 25) | |
| K160/ | MEDPRRRTTAPRAKKPSAKRAPTQPSRTRAHAESCGPQRGARSRRAERDGDTTEKPRAPG | N196L/ |
| R161 | PRVHPARATELTKDAQPSAMDAAGATARPAVRVPQQQAILDPELPAVREPQPPADPEARK | T197V/ |
| Reversion | VVRGPSHRRGARSTGQPRAPRGSRKEPDKLKKVLDKLRLKRKDISEAAETVNKVVERLLR | Y200F/ |
| CA- | RMQKRESEFKGVEQLLVGSFYEKVKISAPNEFDVMFKLEVPRIELQEYYETGAFYLVKFK | H203K |
| mcGAS- | RIPRGNPLSHFLEGEVLSATKMLSKFRKIIKEEVKEIKDIDVSVEKEKPGSPAVTLLIRN | |
| 41 | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 26) | |
| K160 | MEDPRRRTTAPRAKKPSAKRAPTQPSRTRAHAESCGPQRGARSRRAERDGDTTEKPRAPG | R161P/ |
| Reversion | PRVHPARATELTKDAQPSAMDAAGATARPAVRVPQQQAILDPELPAVREPQPPADPEARK | N196L/ |
| CA- | VVRGPSHRRGARSTGQPRAPRGSRKEPDKLKKVLDKLRLKPKDISEAAETVNKVVERLLR | T197V/ |
| mcGAS- | RMQKRESEFKGVEQLLVGSFYEKVKISAPNEFDVMFKLEVPRIELQEYYETGAFYLVKFK | Y200F/ |
| 41 | RIPRGNPLSHFLEGEVLSATKMLSKFRKIIKEEVKEIKDIDVSVEKEKPGSPAVTLLIRN | H203K |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 27) | |
| R161 | MEDPRRRTTAPRAKKPSAKRAPTQPSRTRAHAESCGPQRGARSRRAERDGDTTEKPRAPG | K160D/ |
| Reversion | PRVHPARATELTKDAQPSAMDAAGATARPAVRVPQQQAILDPELPAVREPQPPADPEARK | N196L/ |
| CA- | VVRGPSHRRGARSTGQPRAPRGSRKEPDKLKKVLDKLRLDRKDISEAAETVNKVVERLLR | T197V/ |
| mcGAS- | RMQKRESEFKGVEQLLVGSFYEKVKISAPNEFDVMFKLEVPRIELQEYYETGAFYLVKFK | Y200F/ |
| 41 | RIPRGNPLSHFLEGEVLSATKMLSKFRKIIKEEVKEIKDIDVSVEKEKPGSPAVTLLIRN | H203K |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 28) | |
| L159I/ | MEDPRRRTTAPRAKKPSAKRAPTQPSRTRAHAESCGPQRGARSRRAERDGDTTEKPRAPG | L159I/ |
| K160P/ | PRVHPARATELTKDAQPSAMDAAGATARPAVRVPQQQAILDPELPAVREPQPPADPEARK | K160P/ |
| R161A- | VVRGPSHRRGARSTGQPRAPRGSRKEPDKLKKVLDKLRIPAKDISEAAETVNKVVERLLR | R161A/ |
| CA- | RMQKRESEFKGVEQLLVGSFYEKVKISAPNEFDVMFKSEVPRIELQEYYETGAFYLVKFK | N196L/ |
| CGAS- | RIPRGNPLSHFLEGEVLSATKMLSKFRKIIKEEVKEIKDIDVSVEKEKPGSPAVTLLIRN | T197V/ |
| 41 | PEEISVDIILALESKGSWPISTKEGLPIQGWLGTKVRTNLRREPFYLVPKNAKDGNSFQG | Y200F |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 29) | |
| K160P/ | MEDPRRRTTAPRAKKPSAKRAPTQPSRTRAHAESCGPQRGARSRRAERDGDTTEKPRAPG | K160P/ |
| R161A- | PRVHPARATELTKDAQPSAMDAAGATARPAVRVPQQQAILDPELPAVREPQPPADPEARK | R161A/ |
| CA- | VVRGPSHRRGARSTGQPRAPRGSRKEPDKLKKVLDKLRLPAKDISEAAETVNKVVERLLR | N196L/ |
| CGAS- | RMQKRESEFKGVEQLLVGSFYEKVKISAPNEFDVMFKLEVPRIELQEYYETGAFYLVKEK | T197V/ |
| 41 | RIPRGNPLSHFLEGEVLSATKMLSKFRKIIKEEVKEIKDIDVSVEKEKPGSPAVILLIRN | Y200F |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 30) | |
| K160P- | MEDPRRRTTAPRAKKPSAKRAPTQPSRTRAHAESCGPQRGARSRRAERDGDTTEKPRAPG | K160P/ |
| CA- | PRVHPARATELTKDAQPSAMDAAGATARPAVRVPQQQAILDPELPAVREPQPPADPEARK | N196L/ |
| CGAS- | VVRGPSHRRGARSTGQPRAPRGSRKEPDKLKKVLDKLRLPRKDISEAAETVNKVVERLLR | T197V/ |
| 41 | RMQKRESEFKGVEQLLVGSFYEKVKISAPNEFDVMFKLEVPRIELQEYYETGAFYLVKFK | Y200F |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 31) | |
| CA- | MEDPRRRTTAPRAKKPSAKRAPTQPSRTRAHAESCGPQRGARSRRAERDGDTTEKPRAPG | R158S/ |
| CGAS- | PRVHPARATELTKDAQPSAMDAAGATARPAVRVPQQQAILDPELPAVREPQPPADPEARK | K160D/ |
| 41 | VVRGPSHRRGARSTGQPRAPRGSRKEPDKLKKVLDKLSLDPKDISEAAETVNKVVERLLR | R161P/ |
| R158S | RMQKRESEFKGVEQLLVGSFYEKVKISAPNEFDVMFKLEVPRIELQEYYETGAFYLVKFK | N196L/ |
| | RIPRGNPLSHFLEGEVLSATKMLSKFRKIIKEEVKEIKDIDVSVEKEKPGSPAVTLLIRN | T197V/ |
| | PEEISVDIILALESKGSWPISTKEGLPIQGWLGTKVRTNLRREPFYLVPKNAKDGNSFQG | Y200F |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 32) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| T197I | RMQKRESEFKGVEQLLIGSFYEKVKISAPNEFDVMFKLEVPRIELQEYYETGAFYLVKFK | T197I/ |
| | RIPRGNPLSHFLEGEVLSATKMLSKFRKIIKEEVKEIKDIDVSVEKEKPGSPAVILLIRN | Y200F/ |
| | PEEISVDIILALESKGSWPISTKEGLPIQGWLGTKVRTNLRREPFYLVPKNAKDGNSFQG | H203K |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 33) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| Y200I | RMQKRESEFKGVEQLLVGSIYEKVKISAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200I/ |
| | PEEISVDIILALESKGSWPISTKEGLPIQGWLGTKVRTNLRREPFYLVPKNAKDGNSFQG | H203K |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 34) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| T197I | RMQKRESEFKGVEQLLIGSIYEKVKISAPNEFDVMFKLEVPRIELQEYYETGAFYLVKFK | T197I/ |
| Y200I | RIPRGNPLSHFLEGEVLSATKMLSKFRKIIKEEVKEIKDIDVSVEKEKPGSPAVTLLIRN | Y200I/ |
| | PEEISVDIILALESKGSWPISTKEGLPIQGWLGTKVRTNLRREPFYLVPKNAKDGNSFQG | H203K |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 35) | |
| CA- | MEDPRRRTTAPRAKKPSAKRAPTQPSRTRAHAESCGPQRGARSRRAERDGDTTEKPRAPG | K160D/ |
| cGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| S207G | RMQKRESEFKGVEQLLVGSFYEKVKIGAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVILLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | S207G |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 36) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| S207I | RMQKRESEFKGVEQLLVGSFYEKVKIIAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | S207I |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 37) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| S207V | RMQKRESEFKGVEQLLVGSFYEKVKIVAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | S207V |
| | YHVKTAIFHMWTQDPQDSQWDPRNLSSCFDKLLAFFLECLRTEKLDHYFIPKFNLFSQEL | |
| | IDRKSKEFLSKKIEYERNNGGPIFDKL (SEQ ID NO: 38) | |
| WT | MEDPRRRTTAPRAKKPSAKRAPTQPSRTRAHAESCGPQRGARSRRAERDGDTTEKPRAPG | Y201E |
| Mouse | PRVHPARATELTKDAQPSAMDAAGATARPAVRVPQQQAILDPELPAVREPQPPADPEARK | |
| Y201E | VVRGPSHRRGARSTGQPRAPRGSRKEPDKLKKVLDKLRLKRKDISEAAETVNKVVERLLR | |
| | RMQKRESEFKGVEQLNTGSYEEHVKISAPNEFDVMFKLEVPRIELQEYYETGAFYLVKFK | |
| | RIPRGNPLSHFLEGEVLSATKMLSKFRKIIKEEVKEIKDIDVSVEKEKPGSPAVILLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRKECLKLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 39) | |
| WT | MEDPRRRTTAPRAKKPSAKRAPTQPSRTRAHAESCGPQRGARSRRAERDGDTTEKPRAPG | Y201F |
| Mouse | PRVHPARATELTKDAQPSAMDAAGATARPAVRVPQQQAILDPELPAVREPQPPADPEARK | |
| Y201F | VVRGPSHRRGARSTGQPRAPRGSRKEPDKLKKVLDKLRLKRKDISEAAETVNKVVERLLR | |
| | RMQKRESEFKGVEQLNTGSYFEHVKISAPNEFDVMFKLEVPRIELQEYYETGAFYLVKFK | |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRKECSKLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 40) | |
| WT | MEDPRRRTTAPRAKKPSAKRAPTQPSRTRAHAESCGPQRGARSRRAERDGDTTEKPRAPG | Y201W |
| Mouse | PRVHPARATELTKDAQPSAMDAAGATARPAVRVPQQQAILDPELPAVREPQPPADPEARK | |
| Y201W | VVRGPSHRRGARSTGQPRAPRGSRKEPDKLKKVLDKLRLKRKDISEAAETVNKVVERLLR | |
| | RMQKRESEFKGVEQLNTGSYWEHVKISAPNEFDVMFKLEVPRIELQEYYETGAFYLVKFK | |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRKECLKLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 41) | |
| K395M/ | MEDPRRRTTAPRAKKPSAKRAPTQPSRTRAHAESCGPQRGARSRRAERDGDTTEKPRAPG | Y201E |
| K399M | PRVHPARATELTKDAQPSAMDAAGATARPAVRVPQQQAILDPELPAVREPQPPADPEARK | |
| mCGAS | VVRGPSHRRGARSTGQPRAPRGSRKEPDKLKKVLDKLRLKRKDISEAAETVNKVVERLLR | |
| Y201E | RMQKRESEFKGVEQLNTGSYEEHVKISAPNEFDVMFKLEVPRIELQEYYETGAFYLVKFK | |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 42) | |
| K395M/ | MEDPRRRTTAPRAKKPSAKRAPTQPSRTRAHAESCGPQRGARSRRAERDGDTTEKPRAPG | Y201F |
| K399M | PRVHPARATELTKDAQPSAMDAAGATARPAVRVPQQQAILDPELPAVREPQPPADPEARK | |
| mCGAS | VVRGPSHRRGARSTGQPRAPRGSRKEPDKLKKVLDKLRLKRKDISEAAETVNKVVERLLR | |
| Y201F | RMQKRESEFKGVEQLNTGSYFEHVKISAPNEFDVMFKLEVPRIELQEYYETGAFYLVKFK | |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 43) | |
| K395M/ | MEDPRRRTTAPRAKKPSAKRAPTQPSRTRAHAESCGPQRGARSRRAERDGDTTEKPRAPG | Y201W |
| K399M | PRVHPARATELTKDAQPSAMDAAGATARPAVRVPQQQAILDPELPAVREPQPPADPEARK | |
| mCGAS | VVRGPSHRRGARSTGQPRAPRGSRKEPDKLKKVLDKLRLKRKDISEAAETVNKVVERLLR | |
| Y201W | RMQKRESEFKGVEQLNTGSYWEHVKISAPNEFDVMFKSEVPRIELQEYYETGAFYLVKFK | |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 44) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 04 | VVRGPSHRRGARSTGQPRAPRGSRKEPDKLKKVLDKLRLDPKDISEAAETVNKVVERLLR | Y201E |
| Y201E | RMQKRESEFKGVEQLNTGSYEEHVKISAPNEFDVMFKLEVPRIELQEYYETGAFYLVKFK | |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 45) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 04 | VVRGPSHRRGARSTGQPRAPRGSRKEPDKLKKVLDKLRLDPKDISEAAETVNKVVERLLR | Y201F |
| Y201F | RMQKRESEFKGVEQLNTGSYFEHVKISAPNEFDVMFKLEVPRIELQEYYETGAFYLVKFK | |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 46) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 04 | VVRGPSHRRGARSTGQPRAPRGSRKEPDKLKKVLDKLRLDPKDISEAAETVNKVVERLLR | Y201W |
| Y201W | RMQKRESEFKGVEQLNTGSYWEHVKISAPNEFDVMFKLEVPRIELQEYYETGAFYLVKFK | |
| | RIPRGNPLSHFLEGEVLSATKMLSKFRKIIKEEVKEIKDIDVSVEKEKPGSPAVTLLIRN | |
| | PEEISVDIILALESKGSWPISTKEGLPIQGWLGTKVRTNLRREPFYLVPKNAKDGNSFQG | |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 47) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 22 | VVRGPSHRRGARSTGQPRAPRGSRKEPDKLKKVLDKLRLDPKDISEAAETVNKVVERLLR | N196P/ |
| Y201E | RMQKRESEFKGVEQLPVGSFEEKVKISAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201E |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 48) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 22 | VVRGPSHRRGARSTGQPRAPRGSRKEPDKLKKVLDKLRLDPKDISEAAETVNKVVERLLR | N196P/ |
| Y201F | RMQKRESEFKGVEQLPVGSFFEKVKISAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201F |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 49) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 22 | VVRGPSHRRGARSTGQPRAPRGSRKEPDKLKKVLDKLRLDPKDISEAAETVNKVVERLLR | N196P/ |
| Y201W | RMQKRESEFKGVEQLPVGSFWEKVKISAPNEFDVMEKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201W |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 50) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| Y201E | RMQKRESEFKGVEQLLVGSFEEKVKISAPNEGDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201E |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 51) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| Y201F | RMQKRESEFKGVEQLLVGSFFEKVKISAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201F |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 52) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 41 | VVRGPSHRRGARSTGQPRAPRGSRKEPDKLKKVLDKLRLDPKDISEAAETVNKVVERLLR | N196L/ |
| Y201W | RMQKRESEFKGVEQLLVGSFWEKVKISAPNEEDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200F/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201W |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 53) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 42 | VVRGPSHRRGARSTGQPRAPRGSRKEPDKLKKVLDKLRLDPKDISEAAETVNKVVERLLR | N196L/ |
| Y201E | RMQKRESEFKGVEQLLTGSFEEKVKISAPNEFDVMFKLEVPRIELQEYYETGAFYLVKFK | Y200F/ |
| | RIPRGNPLSHFLEGEVLSATKMLSKFRKIIKEEVKEIKDIDVSVEKEKPGSPAVTLLIRN | H203K/ |
| | PEEISVDIILALESKGSWPISTKEGLPIQGWLGTKVRTNLRREPFYLVPKNAKDGNSFQG | Y201E |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 54) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 42 | VVRGPSHRRGARSTGQPRAPRGSRKEPDKLKKVLDKLRLDPKDISEAAETVNKVVERLLR | N196L/ |
| Y201F | RMQKRESEFKGVEQLLTGSFFEKVKISAPNEFDVMFKLEVPRIELQEYYETGAFYLVKFK | Y200F/ |
| | RIPRGNPLSHFLEGEVLSATKMLSKFRKIIKEEVKEIKDIDVSVEKEKPGSPAVTLLIRN | H203K/ |
| | PEEISVDIILALESKGSWPISTKEGLPIQGWLGTKVRTNLRREPFYLVPKNAKDGNSFQG | Y201F |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 55) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 42 | VVRGPSHRRGARSTGQPRAPRGSRKEPDKLKKVLDKLRLDPKDISEAAETVNKVVERLLR | N196L/ |
| Y201W | RMQKRESEFKGVEQLLTGSFWEKVKISAPNEFDVMFKLEVPRIELQEYYETGAFYLVKFK | Y200F/ |
| | RIPRGNPLSHFLEGEVLSATKMLSKFRKIIKEEVKEIKDIDVSVEKEKPGSPAVTLLIRN | H203K/ |
| | PEEISVDIILALESKGSWPISTKEGLPIQGWLGTKVRTNLRREPFYLVPKNAKDGNSFQG | Y201W |
| | ETWRLSFSHTEKYILNNHGIEKTCCESSGAKCCRMECLMLMKYLLEQLKKEFQELDAFCS | |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 56) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 50 | VVRGPSHRRGARSTGQPRAPRGSRKEPDKLKKVLDKLRLDPKDISEAAETVNKVVERLLR | N196F/ |
| Y201E | RMQKRESEFKGVEQLFVGSIEEKVKISAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200I/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201E |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 57) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 50 | VVRGPSHRRGARSTGQPRAPRGSRKEPDKLKKVLDKLRLDPKDISEAAETVNKVVERLLR | N196F/ |
| Y201F | RMQKRESEFKGVEQLFVGSIFEKVKISAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200I/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201F |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 58) | |
| CA- | MEDPRRRTTAPRAKKPSAKRAPTQPSRTRAHAESCGPQRGARSRRAERDGDTTEKPRAPG | K160D/ |
| CGAS- | PRVHPARATELTKDAQPSAMDAAGATARPAVRVPQQQAILDPELPAVREPQPPADPEARK | R161P/ |
| 50 | VVRGPSHRRGARSTGQPRAPRGSRKEPDKLKKVLDKLRLDPKDISEAAETVNKVVERLLR | N196F/ |
| Y201W | RMQKRESEFKGVEQLFVGSIWEKVKISAPNEFDVMFKLEVPRIELQEYYETGAFYLVKFK | T197V/ |
| | RIPRGNPLSHFLEGEVLSATKMLSKFRKIIKEEVKEIKDIDVSVEKEKPGSPAVTLLIRN | Y200I/ |
| | PEEISVDIILALESKGSWPISTKEGLPIQGWLGTKVRTNLRREPFYLVPKNAKDGNSFQG | H203K/ |
| | ETWRLSFSHTEKYILNNHGIEKTCCESSGAKCCRMECLMLMKYLLEQLKKEFQELDAFCS | Y201W |
| | YHVKTAIFHMWTQDPQDSQWDPRNLSSCFDKLLAFFLECLRTEKLDHYFIPKFNLFSQEL | |
| | IDRKSKEFLSKKIEYERNNGFPIFDKL (SEQ ID NO: 59) | |

In an embodiment of all aspects and embodiments of the polypeptides disclosed herein, a given amino acid at a non-specified residue can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that the desired activity is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The polypeptides of all aspects and embodiments of the disclosure may further comprise additional amino acid residues at the C-terminus and/or N-terminus. Any additional residues or functional domains may be added as appropriate for an intended purpose. In various non-limiting embodiments, the polypeptides may further comprise one or more functional domains (including but not limited to a therapeutic polypeptide domain, a targeting domain, a diagnostic polypeptide domain, etc.), detectable sequences (ex: fluorescent domains), purification tags, etc.

In another embodiment, compositions are provided comprising 2, 3, 4, 5, 6, 7, 8, 9, 10, or more polypeptides of the disclosure.

In another embodiment, the disclosure provides nucleic acids encoding the polypeptide of any embodiment or combination of embodiments herein. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In one embodiment, the disclosure provides expression vectors comprising a nucleic acid of the disclosure operatively linked to a suitable control sequence. “Expression vector” includes vectors that operatively link a nucleic acid coding region or gene to any control sequences capable of effecting expression of the gene product. “Control sequences” operatively linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another embodiment, the disclosure provides host cell comprising the polypeptide, composition, nucleic acid, or expression vector of embodiment or combination of embodiments of the disclosure. The host cells can be either prokaryotic or eukaryotic, and may transiently or constitutively comprise the polypeptide, composition, nucleic acid, or expression vector.

In a further embodiment, pharmaceutical compositions are provided comprising the polypeptide, composition, nucleic acid, expression vector, and/or host cell of any embodiment or combination of embodiments disclosed herein, and a pharmaceutically acceptable carrier. The pharmaceutical compositions of the disclosure can be used, for example, in the methods of the disclosure described herein. The pharmaceutical composition may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

In another embodiment, the disclosure provides methods and uses of polypeptide, composition, nucleic acid, expression vector, or host cell of any preceding claim for any therapeutic use, including but not limited for use as an adjuvant. The polypeptides disclosed herein are DNA-independent constitutively active Cyclic GMP-AMP Synthase (cGAS) mutants), as disclosed in detail in the examples that follow, and thus the polypeptides, compositions, nucleic acids, expression vectors, or host cells can be used, for example, as adjuvants for vaccine administration and to stimulate anti-tumor immunity.

### Examples

### Abstract

Cyclic GMP-AMP Synthase (cGAS) is a pattern recognition receptor critical for the innate immune response to intracellular pathogens, DNA damage, tumorigenesis, and senescence. Binding to double-stranded DNA (dsDNA) induces conformational changes in cGAS that activate the enzyme to produce 2′-3′ cyclic GMP-AMP (cGAMP), a second messenger that initiates a potent interferon response through its receptor, STING. The cGAS-STING pathway is a major target for prevention and treatment of infectious disease, cancer, and autoimmunity, although most current efforts are limited to small molecule drugs. Here, we combined two-state computational design with informatics-guided design to create constitutively active, dsDNA ligand-independent variants of cGAS (CA-cGAS). We identified CA-cGAS mutants with interferon-stimulating activity approaching that of dsDNA-stimulated wild-type cGAS. DNA-independent adoption of the active conformation by CA-cGAS was directly confirmed by X-ray crystallography. Inducible expression of CA-cGAS in tumor cells in vivo resulted in STING-dependent tumor regression, demonstrating that the designed proteins have therapeutically relevant biological activity. Our work provides a general framework for stabilizing active conformations of enzymes through two-state computational design and provides CA-cGAS variants that could be useful as genetically-encoded adjuvants and as research tools for understanding inflammatory diseases.

### Introduction

The ability to induce a controlled and robust innate immune response is highly desirable for prophylactic and therapeutic immunopotentiation. The cGAS-STING pathway has rapidly become a promising target, with numerous small molecule STING agonists in development, several of which have entered clinical trials. However, several first-generation molecules suffered from low efficacy or adverse reactions. Constitutively active cGAS (CA-cGAS) variants that are ligand-independent could be useful as therapeutic agents because their activity would not depend on factors such as the cell cycle, subcellular localization, or disease state.

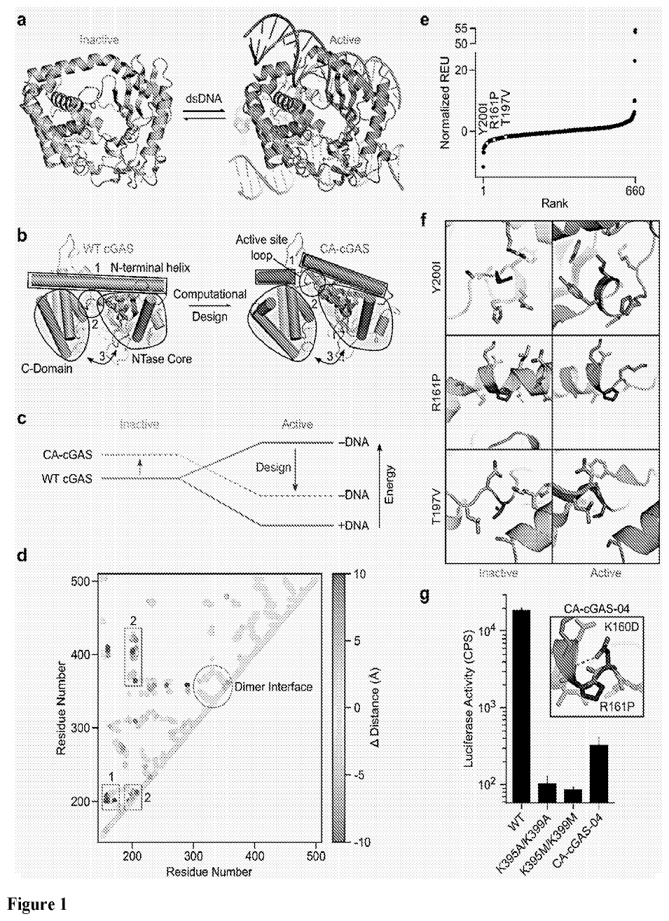

cGAS is a member of the OAS-family, a conserved group of metazoan proteins responsible for double stranded nucleic acid surveillance in the cytosol. OAS family proteins are characterized by an extended N-terminal α-helix, designated the “spine,” a mixed a/P nucleotidyltransferase fold domain (NTase core), and an all α-helical C-terminal domain. cGAS recognizes and is allosterically activated by dsDNA in a sequence-independent manner (FIG. 1a), primarily by forming extensive polar contacts with the phosphate backbone. Although somewhat independent of dsDNA length, activation occurs most efficiently when cGAS binds two strands of dsDNA in roughly parallel orientation, forming long DNA-protein ladders. One dsDNA strand makes extensive contacts along the spine helix, NTase core, and C-domain; while the other dsDNA strand only contacts the NTase core. Multiple structural studies have shown that recognition of dsDNA initiates three changes within the cGAS structure: 1) a break forms in the spine helix (residues 158-161 in murine cGAS [mcGAS]), 2) the dynamic active site loop (residues 196-210) becomes significantly more ordered such that it no longer occludes the active site, and 3) the NTase-core domain moves approximately 3.8 Å closer to the C-domain. The cGAS-DNA complex also contains cGAS-cGAS contacts, and activation by short dsDNA fragments forms cGAS dimers. The sequence of these events and the necessity of each for activation are not fully understood. In contrast to these rearrangements between DNA-free and DNA-bound cGAS, elegant crystallographic studies suggest no significant structural changes during catalysis. These observations indicate that the DNA-bound conformation can be considered the enzymatically active conformation.

We hypothesized that mutations which i) stabilize the dsDNA-bound (active) conformation or ii) destabilize the unbound (inactive) conformation would shift the conformational equilibrium of the enzyme towards the active state, an outcome usually achieved by dsDNA binding (FIG. 1b). Unlike the rearrangements caused by dsDNA binding, the energetic effects of such mutations would be intrinsic to the designed protein, leading to constitutive, dsDNA-independent enzymatic activity (FIG. 1c). We developed a two-state design approach to identify such mutations and generate CA-cGAS variants. The typical protein design approach seeks to optimize the protein sequence for a single target structure, while disregarding or imperfectly approximating the vast space of all other possible states. In contrast, multi-state design seeks to optimize the protein sequence for or against multiple conformations simultaneously. A generalized solution to multi-state protein design that contrasts multiple conformations has been developed, but is complex to use and computationally intensive (19). Here we developed a simple, knowledge-based two-state design protocol that can be generally applied to stabilize specific conformations of dynamic proteins where target and off-target structures are known.

To identify key residues involved in the structural rearrangements between DNA-free and DNA bound conformations, we calculated a distance difference matrix between the DNA-bound (active; PDB ID 4K97) and -unbound (inactive; PDB ID 4K8V) forms of mcGAS. The matrix measures the change in distance for each residue-residue pair in the two structures. Our analysis highlighted two regions that move significantly and are likely critical for cGAS activation: the spine helix above the active site that bridges the NTase core and C-domain (region 1, residues 155-170), and the active site loop and flanking beta strands (region 2, residues 196-214) (FIG. 1b,d). The dimer interface does not undergo any significant structural rearrangements (FIG. 1d).

To identify potential activating mutations in these regions, we evaluated the energy of all possible single amino acid substitutions in both the active and inactive conformations using Rosetta™ (660 mutations total) (20). Mutations were ranked by taking the difference in the energetic impact of each mutation between the active and inactive states, where the energetic impact is defined as the difference between the WT energy and single-mutant energy for a given state (FIG. 1e). This score metric is the central feature of our two-state design approach, as it explicitly favors mutations that stabilize the active conformation, destabilize the inactive conformation, or both. Accordingly, the top-scoring mutations generally fell into two categories: hydrophobic residues exposed to solvent in the inactive conformation and buried in the active conformation (e.g., T197V and Y200I), or proline residues compatible with the backbone geometry of the active conformation but unfavorable in the inactive conformation (e.g., R161P) (FIG. 1f). To generate candidate CA-cGAS designs, combinations of positions with favorable mutations were designed with Rosetta™. Independent design trajectories either restricted the possible mutations at each position to those that scored well in the initial screen, or allowed them to mutate freely. We scored these variants in the same way as the individual mutations, and selected 45 designs containing 1-6 mutations for experimental characterization (Table 5).

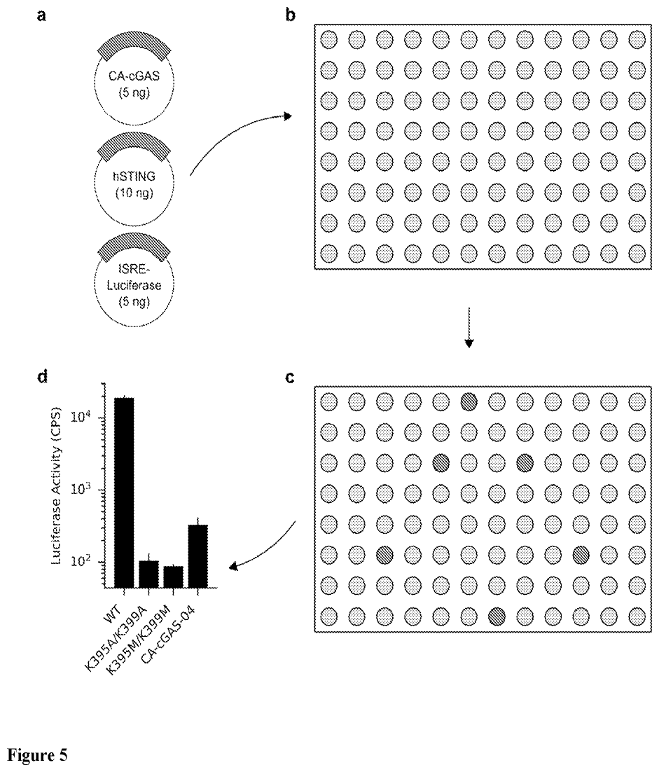

We screened the enzymatic activity of each cGAS variant in HEK293T cells, which do not naturally express cGAS, by transfecting each cGAS construct together with a STING expression vector and a luciferase-based IFN-stimulated response element (ISRE) reporter plasmid (FIG. 5, a-c). To ensure that any measured activity was due to constitutive activation of the enzyme and not activation by transfected plasmid DNA. we first knocked out cGAS activity by disrupting DNA binding through introduction of the previously published K395A/K399A mutations (12). However, because we were concerned that removing the packing provided by the aliphatic portions of these side chains may alter the structure or dynamics of the nearby active site loop, we also confirmed that a K395M/K399M double mutant that retains these packing interactions was equally inactive (FIG. 1g). The 45 best-scoring computationally designed variants were introduced into this K395M/K399M background. In the initial screen, the double mutant K160D/R161P (CA-cGAS-04) exhibited activity higher than the K395M/K399M mutant, but lower than wild-type (WT) cGAS (FIG. 1g). These mutations were intended to destabilize the inactive conformation by breaking the spine helix (R161P) and stabilize the active conformation by capping the helix spanning residues 161-182 (K160D).

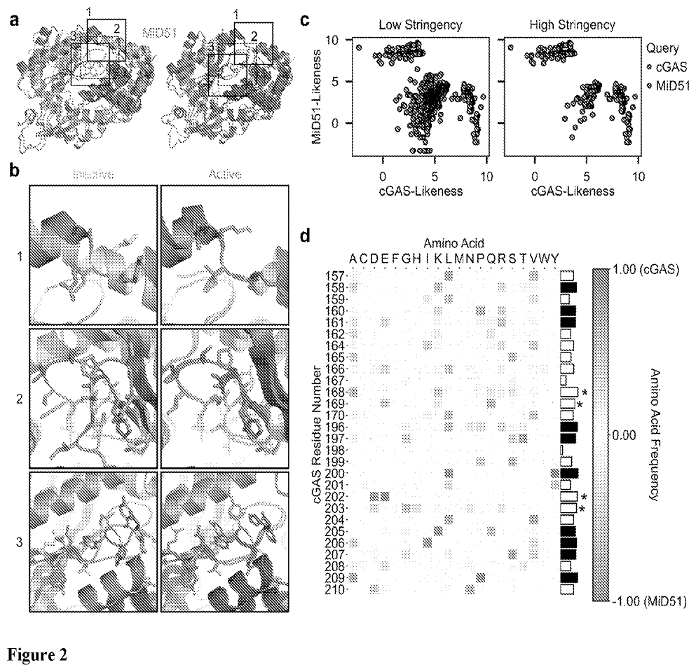

To close the activity gap between CA-cGAS-04 and WT cGAS, we used a combination of bioinformatics and computational approaches. We reasoned that proteins with structural homology specific to the active conformation of cGAS may inspire additional active conformation-stabilizing mutations. We used Protein Data Bank in Europe (21) to search for distantly related proteins with structures closely matching the active conformation of cGAS, and then reviewed available structural and functional data to identify those that are not known to undergo large conformational changes. One protein that matched these criteria was MiD51, an ADP-binding mitochondrial receptor that facilitates mitochondrial fission and adopts the same structure in ADP-bound and unliganded crystal forms (22). MiD51 contains an NTase domain that aligns well with active cGAS (1.96 Å Ca RMSD) (FIG. 2a), though, like many proteins containing NTase domains, it is not an enzyme. The ADP binding site loop and surrounding areas align poorly with the inactive conformation of cGAS, but very well with the active conformation (3.54 Å and 0.34 Å Ca RMSD, respectively; FIG. 2b). Despite this high structural similarity, MiD51 and cGAS have only 18% sequence identity across the aligned domains. Since cGAS undergoes large structural rearrangements upon DNA binding, whereas MiD51 appears to adopt a single static conformation, strongly conserved amino acid differences between the two protein families in cGAS regions 1 and 2 (FIG. 1d) may be related to the functional requirement for movement in cGAS. We hypothesized that mutating these positions in CA-cGAS-04 to the amino acids observed in MiD51-like proteins would further stabilize the active conformation or destabilize the inactive conformation.

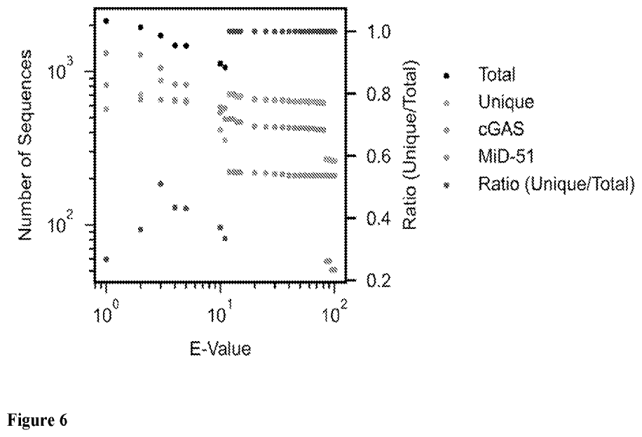

To identify such mutations, we generated multiple sequence alignments (MSAs) of varying permissiveness for cGAS and MiD51 using the hhblits algorithm (23). At low stringency many sequences appeared in both alignments, in keeping with the distant homology between the two proteins, but cGAS-like and MiD51-like clusters were apparent (FIG. 2c). Using a stringent E-value cutoff resulted in a cGAS-like MSA with 468 members and a MiD51-like MSA with 219 members, with no overlap (FIG. 2c and FIG. 6). From these two MSAs we calculated the difference in amino acid frequency at each position, as well as a scaled entropy difference that highlights positions with strongly conserved differences between the two families (FIG. 2d). These data identified target positions and amino acid identities likely to stabilize the active conformation of cGAS. Within the regions specified for computational design, 19 positions have strongly conserved differences. The differences at several positions can be explained by the distinct binding or catalytic functions of MiD51 and cGAS or would not be expected to substantially affect cGAS conformational dynamics; these positions were excluded from further analysis. Ten positions remained: 158, 160, 161, 196, 197, 200, 205, 206, 207, and 209. Of these, residue 160 in mcGAS is a conserved lysine or arginine (combined p=0.70) that binds dsDNA. This position is also conserved in MiD51, but as a proline (p=0.70), which in cGAS would be expected to strongly disfavor formation of the spine helix observed in the inactive conformation, like the R161P mutation in CA-cGAS-04. Residues 196 and 200 in cGAS feature polar amino acids that are partially buried in the active conformation and solvent-exposed in the inactive conformation. Both positions are hydrophobic and well-packed in MiD51, suggesting that they may be useful for stabilizing the active conformation of cGAS.

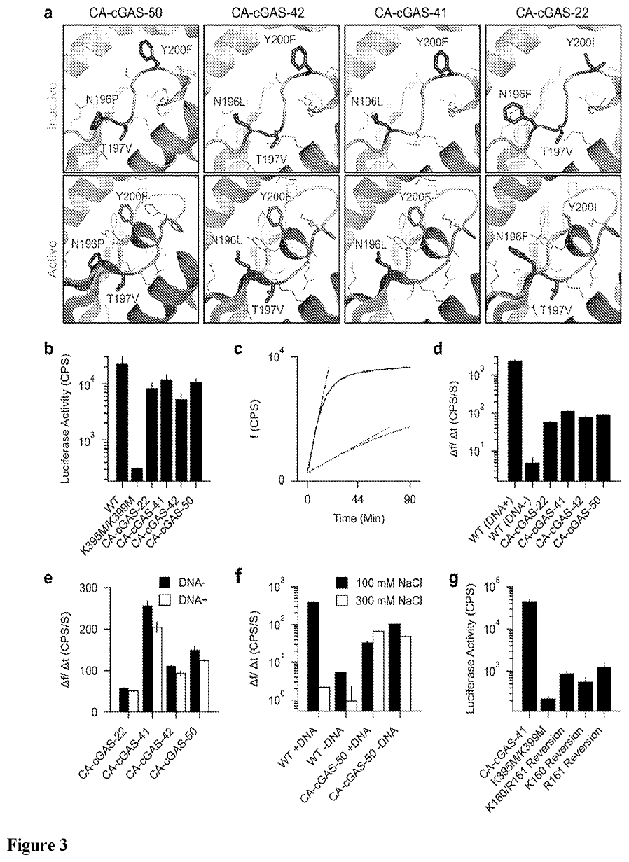

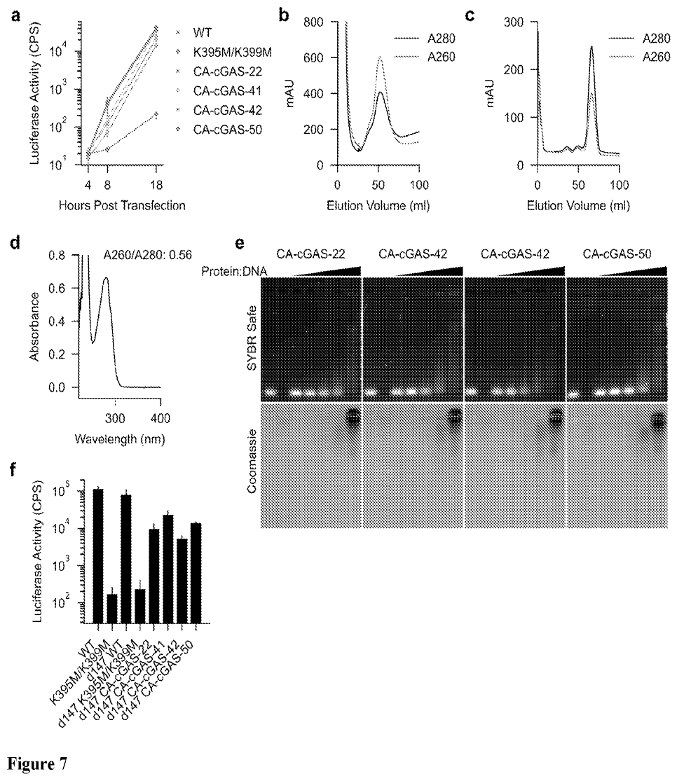

Based on this analysis, a second set of mutants was selected and cloned into the CA-cGAS-04 background. Four of these variants, containing primarily polar to non-polar mutations (FIG. 3a), were constitutively active in the ISRE-luciferase assay (FIG. 3b). Eight hours post-transfection, one variant (CA-cGAS-50) had near-WT levels of activity, while two others (CA-cGAS-22 and CA-cGAS-41) were approximately two-fold lower and CA-cGAS-42 was just above baseline (FIG. 7a). 18 hours post-transfection, the activity of all four variants was near WT (FIG. 7a).

To further characterize these active variants, we expressed and purified the core catalytic domain of each (d147 CA-cGAS), ensuring that our preparations were free of nucleic acid (FIG. 7b), and measured their enzymatic activity under a number of different conditions in vitro. Activity was determined by monitoring the change in fATP fluorescence during the cyclization reaction (FIG. 3b) (15). The most active variant (CA-cGAS-41) was 21-fold less active than WT, and the relative levels of in vitro activity between the variants closely matched that observed by ISRE-luciferase assay in 293T cells (FIG. 3c). Critically, while WT cGAS activity was entirely dependent on the presence of dsDNA, CA-cGAS activity was not (FIGS. 3, c and d). Furthermore, WT cGAS activity is highly salt-dependent, likely due to disruption of dsDNA binding, while CA-cGAS activity is independent of salt concentration (FIG. 3e). To confirm that these CA-cGAS mutants did not bind DNA, we performed an electrophoretic mobility shift assay in which truncated versions of the CA-cGAS variants lacking the unstructured N-terminal domain and a 17 bp dsDNA fragment were mixed at varying molar ratios. No shift was observed until the molar ratio of protein to DNA reached 1:1, and complete shifting was not observed until a ratio of 10:1 (FIG. 7c). Moreover, we confirmed there was no measurable difference in activity between the truncated (d147) and full-length forms of CA-cGAS in cells by the ISRE assay (FIG. 7d). Reverting the CA-cGAS-04 mutations at positions 160 and 161 to WT either individually or together completely eliminated constitutive activity of variant CA-cGAS-41, as measured by ISRE assay (FIG. 3g).

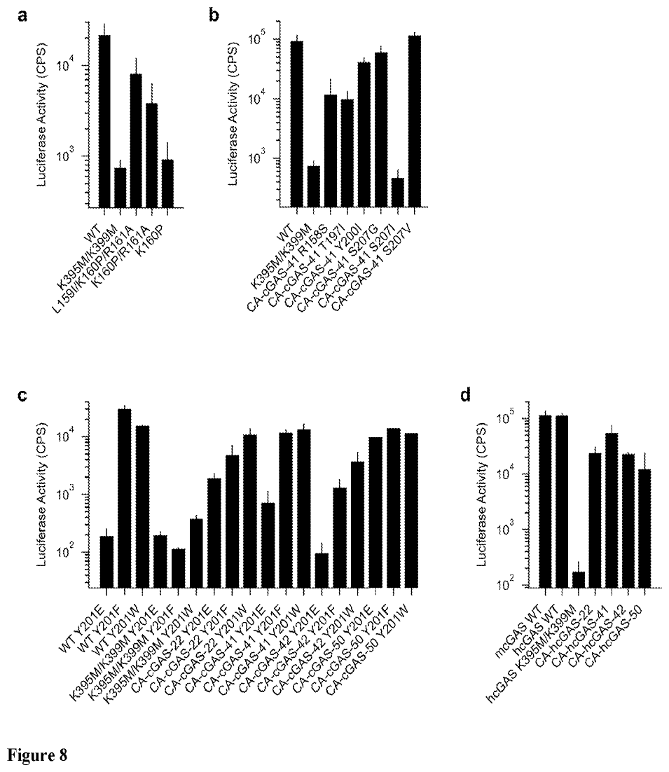

To explore alternative activating mutations in the spine helix, we also characterized a variant of CA-cGAS-41 containing the bioinformatics consensus mutations L159I/K160P/R161A. This variant is as active as CA-cGAS-41 (FIG. 8a). However, K160P/R161A in the same context is less active, and K160P alone is inactive. Additional mutations we predicted may alter CA-cGAS activity had little beneficial effect (FIGS. 8, b and c).

The local environment around regions 1 and 2 in cGAS are reasonably well conserved, which suggests activating mutations should generalize to cGAS from different species. Mutations from CA-cGAS-22, -41, -42, and -50 were introduced with K395M/K399M into human cGAS (hcGAS) and activity was measured by ISRE-luciferase assay. All CA-hcGAS variants had near-WT activity (FIG. 8d). Given that mcGAS and hcGAS share only 55% sequence identity, we expect that the sequence diversity observed in cGAS proteins from other species should have little impact on the effect of the activating mutations described here.

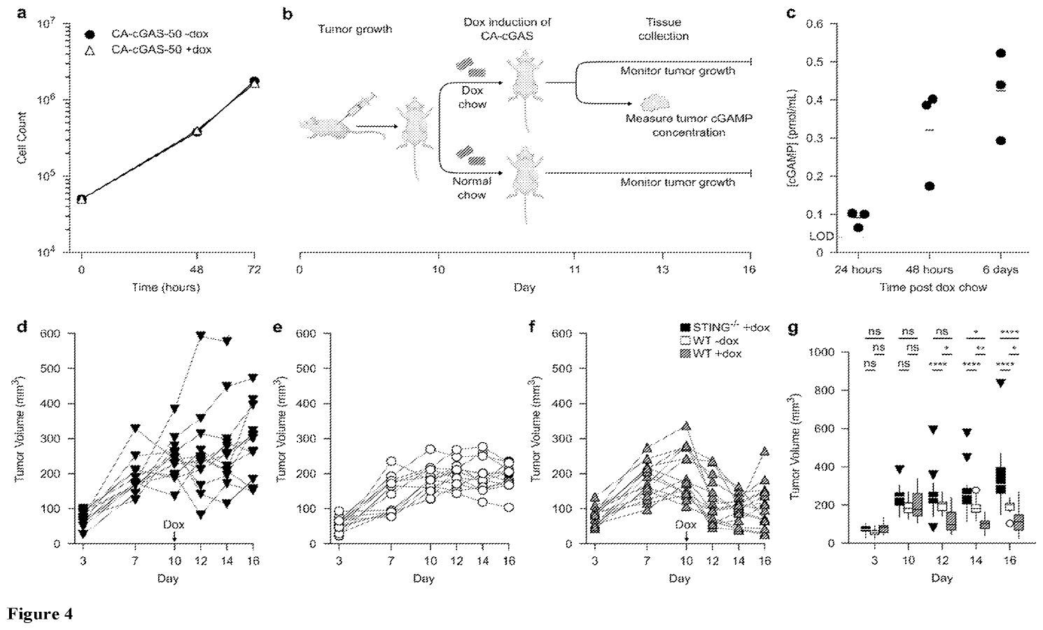

To test the activity and therapeutic potential of CA-cGAS in vivo, we used the B16-F10 mouse melanoma model. Prior studies have shown that intratumoral injection of nuclease-resistant, modified 2′,3′ cyclic di-AMP causes tumor regression, and that this effect requires STING expression in the host, but not the tumor (24-26). This approach is currently being evaluated in humans (NCT02675439 and NCT03172936). Instead of injecting immunostimulatory molecules, we sought to determine whether controlled expression of CA-cGAS could generate therapeutically relevant levels of cGAMP in the tumor itself. To do this, we created a clonal line of B16-F10 cells transduced with a lentivirus encoding doxycycline (dox)-inducible CA-cGAS-50. We found that dox-mediated induction of CA-cGAS-50 did not affect cell growth in vitro (FIG. 4a). We transplanted these cells into WT or Sting^{-/-} mice and allowed the tumors to grow for ten days before placing a cohort of the mice on dox-containing chow (FIG. 4b). We detected robust levels of cGAMP within tumors harvested from mice after dox induction (FIG. 4c). Moreover, we observed significant regression of CA-cGAS-50-expressing tumors in dox-treated WT mice, but not in dox-treated Sting^{-/-} mice (FIG. 4, d-g), demonstrating that CA-cGAS-50 expression in tumor cells was sufficient to activate STING-dependent immunity in surrounding host cells. Together, these data show that CA-cGAS-50 is functional in vivo and could be used as a genetically encoded adjuvant to stimulate anti-tumor immunity.

Our results establish a general multi-state design framework for stabilizing target conformations in structurally dynamic proteins. In brief, this framework has three steps: 1) Identify target and non-target conformations for the system in question, 2) enumerate all possible mutations in dynamic regions in both conformations to identify those with the largest energetic difference between the two states, and 3) combine top-ranked mutations with additional design and bioinformatics analyses based on conformation-specific homologs to identify supporting mutations and prioritize designs for experimental testing. Most current computational design methods typically explicitly consider only a single, static state, which is fundamentally inconsistent with the physical reality of proteins. This is especially true for enzymes and other dynamic systems, making these difficult targets for design. The computational design framework presented herein overcomes many hurdles in multistate design and should be applicable to other multi-state protein design challenges. Our method is also computationally inexpensive and effective even without consideration of possible unknown structural states, such as the unanticipated crystal contacts we observed in our structures. Our designed CA-cGAS variants establish that structural rearrangements alone, without dsDNA binding (12), oligomerization (14, 15), or liquid-liquid phase separation (27), are sufficient for enzyme activity. Their independence of dsDNA binding and therefore the biological status of the cell (e.g., infected vs. uninfected) could make CA-cGAS molecules useful tools for better understanding the biological role of the cGAS-STING pathway in various tissues. Finally, our demonstration that induction of CA-cGAS-50 resulted in STING-dependent tumor regression in vivo establishes CA-cGAS molecules as biologic alternatives to small molecule STING activators with potential prophylactic and therapeutic applications in infectious disease and cancer.

### REFERENCES AND NOTES

1. P. Gao, M. Ascano, Y. Wu, W. Barchet, B. L. Gaffney, T. Zillinger, A. A. Serganov, Y. Liu, R. A. Jones, G. Hartmann, T. Tuschl, D. J. Patel, Cyclic [G(2′,5′)pA(3′,5′)p] is the metazoan second messenger produced by DNA-activated cyclic GMP-AMP synthase. Cell. 153, 1094-1107 (2013).
2. J. Wu, L. Sun, X. Chen, F. Du, H. Shi, C. Chen, Z. J. Chen, Cyclic GMP-AMP is an endogenous second messenger in innate immune signaling by cytosolic DNA. Science. 339, 826-830 (2013).
3. L. Sun, J. Wu, F. Du, X. Chen, Z. J. Chen, Cyclic GMP-AMP synthase is a cytosolic DNA sensor that activates the type I interferon pathway. Science. 339, 786-791 (2013).
4. A. Ablasser, M. Goldeck, T. Cavlar, T. Deimling, G. Witte, I. Rohl, K.-P. Hopfner, J. Ludwig, V. Hornung, cGAS produces a 29-59-linked cyclic dinucleotide second messenger that activates STING. Nature. 498 (2013), doi:10.1038/naturel2306.
5. J. Le Naour, L. Zitvogel, L. Galluzzi, E. Vacchelli, G. Kroemer, Trial watch: STING agonists in cancer therapy. Oncoimmunology. 9, 1777624 (2020).
6. J.-J. Wu, L. Zhao, H.-G. Hu, W.-H. Li, Y.-M. Li, Agonists and inhibitors of the STING pathway: Potential agents for immunotherapy. Med. Res. Rev. 40, 1117-1141 (2020).
7. B. Larkin, V. Ilyukha, M. Sorokin, A. Buzdin, E. Vannier, A. Poltorak, Cutting Edge: Activation of STING in T Cells Induces Type I IFN Responses and Cell Death. J. Immunol. 199, 397-402 (2017).
8. J. T. Bulcha, Y. Wang, H. Ma, P. W. L. Tai, G. Gao, Viral vector platforms within the gene therapy landscape. Signal Transduct Target Ther. 6, 53 (2021).
9. M. D. Buschmann, M. J. Carrasco, S. Alishetty, M. Paige, M. G. Alameh, D. Weissman, Nanomaterial Delivery Systems for mRNA Vaccines. Vaccines (Basel). 9 (2021), doi:10.3390/vaccines9010065.
10. H. E. Volkman, S. Cambier, E. E. Gray, D. B. Stetson, Tight nuclear tethering of cGAS is essential for preventing autoreactivity. Eife. 8 (2019). doi:10.7554/eLife.47491.
11. V. Hornung, R. Hartmann, A. Ablasser, K.-P. Hopfner, OAS proteins and cGAS: unifying concepts in sensing and responding to cytosolic nucleic acids. Nat. Rev. Immunol. 14, 521-528 (2014).
12. F. Civril, T. Deimling, C. C. de Oliveira Mann, A. Ablasser, M. Moldt, G. Witte, V. Hornung, K.-P. Hopfner, Structural mechanism of cytosolic DNA sensing by cGAS. Nature. 498, 332-337 (2013).
13. X. Zhang, J. Wu, F. Du, H. Xu, L. Sun, Z. Chen, C. A. Brautigam, X. Zhang, Z. J. Chen, The cytosolic DNA sensor cGAS forms an oligomeric complex with DNA and undergoes switch-like conformational changes in the activation loop. Cell Rep. 6, 421-430 (2014).
14. X. Li, C. Shu, G. Yi, C. T. Chaton, C. L. Shelton, J. Diao, X. Zuo, C. C. Kao, A. B. Herr, P. Li, Cyclic GMP-AMP Synthase Is Activated by Double-Stranded DNA-Induced Oligomerization. Immunity. 39, 1019-1031 (2013).
15. L. Andreeva, B. Hiller, D. Kostrewa, C. Lassig, C. C. D. O. Mann, D. J. Drexler, A. Maiser, M. Gaidt, H. Leonhardt, V. Homung, K.-P. Hopfner, cGAS senses long and HMGB/TFAM-bound U-turn DNA by forming protein-DNA ladders. Nature (2017), doi:10.1038/nature23890.
16. J. Hall, E. C. Ralph, S. Shanker, H. Wang, L. J. Byrnes, R. Horst, J. Wong, A. Brault, D. Dumlao, J. F. Smith, L. A. Dakin, D. C. Schmitt, J. Trujillo, F. Vincent, M. Griffor, A. E. Aulabaugh, The catalytic mechanism of cyclic GMP-AMP synthase (cGAS) and implications for innate immunity and inhibition. Protein Sci. 26, 2367-2380 (2017).
17. P.-S. Huang, S. E. Boyken, D. Baker, The coming of age of de novo protein design. Nature. 537, 320-327 (2016).
18. B. Kuhlman, G. Dantas, G. C. Ireton, G. Varani, B. L. Stoddard, D. Baker, Design of a novel globular protein fold with atomic-level accuracy. Science. 302, 1364-1368 (2003).
19. A. Leaver-Fay, R. Jacak, P. B. Stranges, B. Kuhlman, A generic program for multistate protein design. PLoS One. 6, e20937 (2011).
20. A. Leaver-Fay, M. Tyka, S. M. Lewis, 0. F. Lange, J. Thompson, R. Jacak, K. Kaufman, P. D. Renfrew, C. A. Smith, W. Sheffler. I. W. Davis, S. Cooper, A. Treuille, D. J. Mandell, F. Richter, Y.-E. A. Ban, S. J. Fleishman, J. E. Coin. D. E. Kim, S. Lyskov, M. Berrondo, S. Mentzer, Z. Popovid, J. J. Havranek, J. Karanicolas, R. Das, J. Meiler, T. Kortemme, J. J. Gray, B. Kuhlman, D. Baker, P. Bradley, ROSETTA3: an object-oriented software suite for the simulation and design of macromolecules. Methods Enzymol. 487, 545-574 (2011).
21. E. Krissinel, K. Henrick, Secondary-structure matching (SSM), a new tool for fast protein structure alignment in three dimensions. Acta Crystallogr. D Biol. Crystallogr. 60, 2256-2268 (2004).
22. V. Richter, C. S. Palmer, L. D. Osellame, A. P. Singh, K. Elgass, D. A. Stroud, H. Sesaki, M. Kvansakul, M. T. Ryan, Structural and functional analysis of mid51, a dynamin receptor required for mitochondrial fission. J. Cell Biol. 204, 477-486 (2014).
23. M. Remmert, A. Biegert, A. Hauser, J. Soding, HHblits: lightning-fast iterative protein sequence searching by HMM-HMM alignment. Nat. Methods. 9, 173-175 (2011).
24. L. Corrales, L. H. Glickman, S. M. McWhirter, D. B. Kanne, K. E. Sivick, G. E. Katibah, S.-R. Woo, E. Lemmens, T. Banda, J. J. Leong, K. Metchette, T. W. Dubensky Jr, T. F. Gajewski, Direct Activation of STING in the Tumor Microenvironment Leads to Potent and Systemic Tumor Regression and Immunity. Cell Rep. 11, 1018-1030 (2015).
25. K. E. Sivick, A. L. Desbien, L. H. Glickman, G. L. Reiner, L. Corrales, N. H. Surh, T. E. Hudson, U. T. Vu, B. J. Francica, T. Banda, G. E. Katibah, D. B. Kanne, J. J. Leong, K. Metchette, J. R. Bruml, C. O. Ndubaku, J. M. McKenna, Y. Feng, L. Zheng, S. L. Bender, C. Y. Cho, M. L. Leong, A. van Elsas, T. W. Dubensky Jr, S. M. McWhirter, Magnitude of Therapeutic STING Activation Determines CD8+ T Cell-Mediated Anti-tumor Immunity. Cell Rep. 25, 3074-3085.e5 (2018).
26. B. J. Francica, A. Ghasemzadeh, A. L. Desbien, D. Theodros, K. E. Sivick, G. L. Reiner, L. Hix Glickman, A. E. Marciscano, A. B. Sharabi, M. L. Leong, S. M. McWhirter, T. W. Dubensky Jr, D. M. Pardoll, C. G. Drake, TNFα and Radioresistant Stromal Cells Are Essential for Therapeutic Efficacy of Cyclic Dinucleotide STING Agonists in Nonimmunogenic Tumors. Cancer Immunol Res. 6, 422-433 (2018).
27. M. Du, Z. J. Chen, DNA-induced liquid phase condensation of cGAS activates innate immune signaling. Science. 1022, 1-10 (2018).
28. Y.-T. K. Lau, V. Baytshtok, T. A. Howard, B. M. Fiala, J. M. Morgan, L. P. Carter. D. Baker, C. D. Lima, C. D. Bahl, Discovery and engineering of enhanced SUMO protease enzymes Downloaded from SUMO protease engineering. 5 (2018), doi:10.1074/jbc.RA118.004146.
29. H. Ishikawa, Z. Ma, G. N. Barber, STING regulates intracellular DNA-mediated, type I interferon-dependent innate immunity. Nature. 461, 788-792 (2009).
30. A. Gall, P. Treuting, K. B. Elkon, Y.-M. Loo, M. Gale Jr, G. N. Barber, D. B. Stetson, Autoimmunity initiates in nonhematopoietic cells and progresses via lymphocytes in an interferon-dependent autoimmune disease. Immunity. 36, 120-131 (2012).
31. A. G. Snyder, N. W. Hubbard, M. N. Messmer, S. B. Kofman, C. E. Hagan, S. L. Orozco, K Chiang, B. P. Daniels, D. Baker, A. Oberst, Intratumoral activation of the necroptotic pathway components RIPK1 and RIPK3 potentiates antitumor immunity. Sci Immunol. 4 (2019), doi:10.1126/sciimmunol.aaw2004.
32. H. Liu, H. Zhang, X. Wu, D. Ma, J. Wu, L. Wang, Y. Jiang, Y. Fei, C. Zhu, R. Tan, P. Jungblut, G. Pei, A. Dorhoi, Q. Yan, F. Zhang. R. Zheng, S. Liu, H. Liang, Z. Liu, H. Yang, J. Chen, P. Wang, T. Tang, W. Peng, Z. Hu, Z. Xu, X. Huang, J. Wang, H. Li, Y. Zhou, F. Liu, D. Yan, S. H. Kaufmann, C. Chen, Z. Mao, B. Ge, Nuclear cGAS suppresses DNA repair and promotes tumorigenesis. Nature (2018). doi:10.1038/s41586-018-0629-6.

### Materials and Methods

### Computational Design

Structures of mcGAS in the active (PDB ID 4K97) and inactive (PDB ID 4K8V) conformations were downloaded from RCSB. The residue-residue distance difference matrix was generated by calculating the alpha-carbon or side-chain heavy atom distances for the active and inactive conformations. The difference is the inactive conformation residue-residue distance subtracted from the active conformation residue-residue distance. To prepare the PDB files for computational design, all heteroatoms and DNA atoms were removed. For PDB 4K8V all chains except chain A were removed. The Rosetta™ computational design methodology was used to introduce point mutants at every residue in regions of the enzyme that undergo putatively relevant conformational changes during activation. Each residue was mutated to every other residue and any residues in a 10 Å sphere were allowed to repack (sample rotamer conformations) to accommodate the mutation. The entire pose was then minimized, but backbone atoms were constrained outside of a 15 Å sphere around the target residue. This protocol was applied to both the active and inactive conformation. The difference in total energy in RosettaTh Energy Units (REU) was calculated for the mutant and WT active conformations (dActive), and for the mutant and WT inactive conformations (dInactive). The difference between dActive and dInactive (normalized REU) was used to rank order mutants. A mutant with negative normalized REU should bias the structure towards the active conformation. The best scoring single mutants were combined or improved by either allowing any other site where a beneficial mutation was observed to mutate to one of those beneficial mutations or allowing design to any residue at those sites. Designs were scored as described above.

### Bioinformatics

We also took a bioinformatics approach to identify likely variants. First we searched the PDB for cGAS structural homologues that had low sequence conservation using the protein structure comparison service PDBeFold at the European Bioinformatics Institute, paying close attention to the alignment of the active site and dynamic regions identified in cGAS. We then performed a literature search for information about the biochemistry of hits, looking specifically for a protein that did not undergo structural rearrangements, and identified a suitable protein, MiD51 (PDB ID 40AF). Using MiD51 and cGAS sequences excluding the unstructured N-terminal domain as inputs, we generated two overlapping multiple sequence alignments (MSAs) using the HHblits algorithm with a high e-value cutoff We then combined the two alignments and calculated bit-scores for cGAS and MiD51. We clustered the bit-score from alignments to cGAS and MiD51 using a Gaussian mixture model with Dirichlet process. We then generated MSAs with increasing e-value cutoff until there was no overlap between the cGAS MSA and MiD51 MSA, resulting in MSAs containing cGAS-like and MiD51-like proteins. We calculated the site-specific amino acid frequencies for each group, and the frequency difference between groups. To evaluate the magnitude of the differences in site specific amino acid frequencies we calculated the divergence. The site specific amino acid frequency divergence between cGAS and MiD51 multiple sequence alignments was calculated from the site specific entropy S=−Σpa×log₂ (pa). The divergence was then calculated according to

$$D = \sqrt{{H}_{mixed} - \frac{{H}_{cGAS} + {H}_{MiD 51}}{2}}$$

where H_{mixed} is calculated from the average frequency

$${pa}_{mixed} = \frac{{pa}_{cGAS} + {pa}_{MiD 51}}{2}$$

where pa is the site specific amino acid frequency. By calculating the difference in site specific amino acid frequency between cGAS-like and MiD51-like proteins, we could identify key regions and mutations that were likely to explain the difference in function between the two proteins, specifically the presence or absence of a conformational change. Using those data we combined sets of single mutations to make constitutively active cGAS variants for experimental testing. Some heuristics were applied when selecting design variants for experimental testing, such as minimizing distance in primary structure between mutations to facilitate primer design for Kunkel mutagenesis. Structural alignments between MiD51 and cGAS were generated in PyMOL with the “super” command applied to the full structure, or for details, by local alignments. The spine helix alignment was made between cGAS (4k97) residues 158-161 and MiD51 residues 148-151. The active site loop alignment was made between cGAS residues 194-203 and MiD51 residues 184-193. The inactive conformation of cGAS (4K8V) was then aligned to the active conformation to compare MiD51 to inactive cGAS.

### Plasmid Preparation

The sequences for mouse cGAS (uniprot: Q8C6L5) and human cGAS (uniprot: Q8N884) were optimized for expression in mammalian cells and purchased as synthetic gBlocks from IDT. The gene fragment was amplified using Phusion™ Polymerase per the manufacturer's instructions with primers incorporating homology regions for the 5′ or 3′ ends of the target cut vector (pCDNA3, pET28b, or pCDB179) and for the gBlock. For pCDNA3.1 and pET28b vectors the primers were homologous to the 5′ and 3′ ends of the gBlock. In some applications the sequence for a myc tag (GAACAGAAACTGATTAGCGAAGAAGAT; SE ID NO:60) was included in the antisense primer. For pCDB179 the sense primer was homologous for the 3′ end of the cut vector, and for the 436th-454th base pairs in the gBlock, corresponding to a truncation at the 148th amino acid. Because cloning into pCDB179 introduces a SUMO domain on the N terminus of the protein and the 148th amino acid is a proline, a non-homologous serine codon (TCG) was added between the primer and gBlock homology regions. When the SUMO domain is cleaved from the peptide, the serine residue remains. Constructs with this truncation will be referred to as d147 cGAS. The antisense primer was homologous for the 5′ cut end of the vector. The resulting amplicons were assembled with restriction-digested (NdeI and XhoI) pET28b, (XhoI and BamHI) pCDB179, or (XhoI and NotI) pCDNA3.1 using Gibson Assembly and transformed into chemically competent E. coli DH5a cells (NEB). Colonies were verified by Sanger sequencing. To identify CA-cGAS, the ability to bind and be activated by DNA was first removed by introducing the mutations K395M and K399M into the wild-type sequence. The mutations were made by site directed mutagenesis. The resulting amplified plasmids were transformed into chemically competent K coil DH5 cells (NEB) and colonies verified by Sanger sequencing.

Activating mutations identified by computational design were generated by site directed mutagenesis using the Kunkel method. Single stranded, uracilated plasmid was generated by transforming genes in pCDNA3 plasmid into chemically competent CJ236 cells and inoculating six colonies into 3 mL of LB with carbenicillin. Cultures were allowed to expand for three to four hours at 37° C., shaking at 200 rpm before adding 3×10⁹ plaque forming units of M13K07 helper phage. After an additional hour the culture was expanded 1.50 and grown overnight at 37° C., shaking at 200 rpm. Phage was isolated from the culture medium by first clarifying the medium and then pelleting the phage in 3.3% PEG 8000, 420 mM NaCl. Single stranded DNA was harvested using a Qiagen Qiaprep™ M13 kit (#27704). Oligonucleotide primers containing the desired mutation or mutations were obtained from IDT. Oligonucleotides were phosphorylated with T4 polynucleotide kinase (NEB) and diluted to an appropriate working concentration. Oligonucleotides were annealed to the single stranded template by slowly lowering the temperature from 95° C. to 25° C. at 1° C./min. The phosphorylated and annealed oligonucleotides were used to prime in vitro DNA synthesis, with T4 DNA ligase and T7 DNA polymerase (NEB). The product, double stranded heteroduplex plasmid, was transformed into chemically competent E. coli DH5a cells (NEB). Colonies were verified by Sanger sequencing.

CA-cGAS genes were cloned from pCDNA3 to pCDB179 or pET28b+ by in vitro amplification with an appropriate set of primers containing homology for the target plasmid, and then Gibson assembled into cut vector. pET28b was cut with XhoI and NdeI. pCDB179 was cut with XhoI and BamH1. For crystallography and in vitro activity assays, a truncated d147 variant was cloned into pCDB179 with a single serine residue inserted between the C-terminal SUMO glycine and N-terminal P147 residue in cGAS. The serine residue facilitates cleavage during purification.

### Cell-Based ISRE Assay

To screen likely constitutively active mutants, pCDNA3 plasmid containing the mutant cGAS gene was purified from chemically competent E. coli DH5a cells (NEB) using a Qiagen Miniprep™ kit. 293T cells were seeded into 96-well flat bottom plates at 2.5×10⁴ per well. Cells were transiently transfected with three plasmids containing mutant cGAS (5 ng/well), STING (20 ng/well), and luciferase under regulation by an Interferon Stimulated Response Element (5 ng/well). The cells were incubated for 8 to 18 hours before being lysed and luciferase activity assessed using the Luciferase Assay System (Promega E4550) according to the manufacturer's instructions.

### Production and Purification

cGAS mutants showing activity by ISRE assay were purified recombinantly from chemically competent E. coli T7 cells (NEB). A small culture of TB with kanamycin was inoculated and grown overnight at 37° C. Cultures were then expanded 1:50 into 500 mL of TB with kanamycin in a 2 L baffled shake flask and grown up to approximately OD 0.6 at 37° C., shaking at 220 rpm. Once the indicated cell density was achieved, expression was induced by the addition of β-D-1-thiogalactopyranoside (IPTG) at a final concentration of 1 mM. Expression proceeded for 18 h at 18° C. with shaking at 220 r.p.m. Cultures were harvested by centrifugation at 5,000 r.c.f. for 10 minutes, and the supernatant discarded. The pellet was resuspended in lysis buffer (50 mM Tris pH 8.0, 300 mM NaCl, 20 mM imidazole, 1 mM PMSF, 1 mM DTT, 0.1 mg/ml DNAse, and 0.1 μM RNase) and lysed at 4° C. by sonication with a probe sonicator at 70% power for two minutes or microfluidization with a single pass at 20,000 psi. Lysate was clarified by centrifugation at 12,000 r.c.f for 30 minutes. Clarified lysate was further filtered through a 0.22 μm PVDF membrane before loading onto a 5 mL HisTrap™ HP (Cytiva Life Sciences) column. The column was washed with approximately 25 mL of running buffer (50 mM Tris pH 8.0, 300 mM NaCl, 20 mM imidazole, 1 mM DTT) at 3 mL per minute and the protein eluted with a linear gradient from 0% to 100% elution buffer (50 mM Tris pH 8.0, 300 mM NaCl, 500 mM imidazole, 1 mM DTT) over 40 minutes. Protein elution was monitored by absorbance at 280 nm and purity estimated by monitoring absorbance at 260 nm.

To remove any bound dsDNA, the major elution fractions were pooled further purified by Heparin affinity chromatography. Pooled fractions were diluted in 20 mM Tris pH 8.0 to bring the final NaCl concentration down to approximately 200 mM. For full-length cGAS constructs the NaCl concentration must not drop below 200 mM, otherwise precipitation occurs. Truncated d147 cGAS constructs are more tolerant of low ionic strength. The diluted protein was then loaded onto a 5 mL HiTrap™ Heparin HP column (Cytiva Life Sciences). Nucleic acids were eluted by washing the column with 25 mL of wash buffer (20 mM Tris pH 8.0, 250 mM NaCl, 1 mM DTT), then the protein was eluted with a linear gradient from 0% to 100% elution buffer (20 mM Tris pH 8.0, 1000 mM NaCl, 1 mM DTT) over 40 minutes. Elution was monitored by absorbance at 260 nm (nucleic acids) and 280 nm (protein).

For d147 cGAS constructs, the SUMO domain was cleaved with a custom Ulpl variant produced as previously described (28). The protein concentration was first estimated by absorbance at 280 nm. After correcting for scattering, the concentration was calculated based on the theoretical molar extinction coefficient, assuming all cysteine residues are reduced. One milligram of protease per every 100 mg cGAS was added directly to the pooled fractions from the heparin column, without modification of the buffer. The cleavage reaction was allowed to proceed overnight at 4° C. The extent of cleavage was followed by SDS-PAGE. To remove SUMO, Ulpl protease, and uncleaved cGAS the entire reaction volume was loaded onto a 5 mL HisTrap™ HP column and the flowthrough collected. The column was then washed with 25 mL of running buffer at pH 7.5 and protein elution monitored at 280 nm. The major wash fractions were pooled with the flow through.

Full length or cleaved d147 cGAS constructs were further purified by SEC. Nickel or Heparin affinity eluates were pooled and concentrated in a spin concentrator to approximately one ml volume or a final concentration of ˜20 mg/mL, whichever was larger. One ml aliquots were injected onto a Superdex™ 75 10/300 GL column. For d147 cGAS constructs the column was equilibrated and run with low salt SEC buffers (20 mM Tris pH 7.5, 100 mM NaCl, 1 mM DTT). If the protein was to be frozen for storage, the buffer included 250 mM glycine and 5% v/v glycerol, except for d147 WT and d147 K395M/K399M cGAS, which was eluted in low salt SEC buffer, without glycine. Full-length constructs were eluted into low-salt SEC buffers. Again, if the protein was to be frozen, it was eluted in buffer with 250 mM glycine and 5% v/v glycerol. except WT and K395M/K399M cGAS, which was always eluted in high-salt (300 mM NaCl) SEC buffer. Full-length constructs eluted at 10.8 mL, and d147 constructs eluted at 12.6 mL. Major fractions were pooled and quantified by absorbance at 280 nm. Presence of DNA was evaluated by 260/280 nm ratio. Protein identity and purity was confirmed by SDS-PAGE, Liquid Chromatography-Mass Spectrometry, and UV-Vis spectroscopy. Constructs were frozen by diluting in SEC buffer to an appropriate concentration and aliquoting into cryo-safe tubes. Tubes were flash frozen in liquid nitrogen and stored at −80° C.

In Vitro cGAS Activity Assay

In vitro cGAS activity was determined using the method previously reported by Andreeva et al. (15). Activity was measured by combining 1 μM cGAS, 500 μM GTP, and 50 μM 2-aminopurine riboside-5′-O-triphosphate (fATP) (Biolog) with or without 2.6 ng/L plasmid dsDNA. The reaction was initiated by spiking in 5 mM MgCl₂. The fluorescence intensity of fATP was monitored for three hours at 32° C. on a Synergy Neo2 plate reader (Biotek), with λₑₓ 305 nm and λₑₘ 363 nm. As cGAS converts GTP and fATP into cyclic GMP-fAMP, the fluorescence intensity decreases. The fluorescence intensity was normalized to a buffer-only sample with 50 μM fATP. The initial rate was fit with a linear model.

### Electrophoretic Mobility Shift Assay

The interaction between CA-cGAS and dsDNA was evaluated by electrophoretic mobility shift. A 1% agarose gel was prepared in 1×Tris/Glycine buffer (Bio-Rad). CA-cGAS was mixed with a 40 bp dsDNA fragment purchased from IDT in 20 mM Tris pH 7.5, 100 mM NaCl buffer. Mixtures were set up such that the final concentration of dsDNA was constant for all ratios. After incubating CA-cGAS and dsDNA mixtures for 30 minutes, 2 μL of glycerol was added to 10 μL of the reaction and loaded into the pre-prepared agarose gel. Gels were run in 1×Tris/Glycine buffer at 50 volts for one hour. The gel was stained for 15 minutes in 1×SYBR™ Gold (Invitrogen) in the dark and imaged with a UV transilluminator. The gel was then stained for protein with GelCode™ Blue Stain Reagent (Thermo Scientific) for 15 minutes, de-stained overnight in water, and imaged.

### Lentiviral Vector

Murine cGAS (mcGAS) mut50 was cloned into the pSLIK™ vector as described (10).

### Cell Culture

B16-F10 cells were maintained in Dulbecco's modification of Eagle medium (DMEM) supplemented with 10% FBS, 2 mM L-glutamine, 10 mM HEPES, 1 mM sodium pyruvate, 0.05 mM beta-mercaptoethanol, 100 IU penicillin, and 100 μg/ml streptomycin (complete DMEM). A clonal line of parental B16-F10 cells was created by lentiviral transduction of a construct that constitutively expressed the mWasabi™ fluorescent protein fused to a nuclear localization signal and a peptide derived from ovalbumin (SIINFEKL). These cells were transduced with pSLIK CA-cGAS-50. Single clones were grown up and tested for expression levels of CA-cGAS-50. A clone with low basal cGAMP production and high inducible expression of CA-cGAS-50 was selected for further experiments. For quantification of in vitro cell growth and CA-cGAS-50 induction, doxycycline was used at 1 g/ml in complete DMEM.

### Mice

C57BL6/J mice were purchased from Jackson and allowed at least one week to acclimate prior to experiment initiation. STING KO mice (Tmem173−/−) were generated as described (29) and backcrossed to C57BL/6J (30). All mice were maintained in a specific pathogen-free (SPF) barrier facility at the University of Washington, and all experiments were done in accordance with the Institutional Animal Care and Use Committee guidelines of the University of Washington.

### Mouse Tumor Implantation

9-13 week old female mice were injected subcutaneously into the flank with 1×105 tumor cells mixed 1:1 with Matrigel™ Basement Matrix (Corning #354248) for a final injection volume of 100 μL. Tumor volume was calculated using the following formula: Volume=Short axis²×Long axis×0.523 (31). Mice receiving doxycycline were switched to doxycycline chow (Envigo #TD.01306 625 mg/kg irradiated) starting at day 10-11 post tumor implantation.

Tumor cGAMP Measurements

Tumors were dissected out of the mice and cut into small pieces in 3 ml of dissociation buffer: 2.7 mg/mL Collagenase A (Sigma #110088793001), 23 U/mL DNaseI (Sigma #D4263-5VL), 2 mM CaCl₂) in 1×PBS. Tumors were then incubated at 37° C. with shaking for 30 minutes. 3 mL of termination buffer (2% FCS, 5 mM EDTA in 1×PBS) was then added. Samples were then filtered through a 70 mm mesh strainer, washed twice in 1×PBS, and lysed in 200 μL RIPA buffer: 150 mM NaCl, 1.0% NP-40 substitute, 0.5% sodium deoxycholate, 0.1% SDS, 50 mM Tris pH 8.0). 50 μL of sample was used per well to measure cGAMP content (Arbor Assays 2′,3′-Cyclic GAMP ELISA Kit #K067-H5W) per the manufacturer's instructions.

### Statistics

Tumor growth and cGAMP quantification data were visualized and analyzed using GraphPad™ Prism software. Statistical tests used to analyze data are noted in the figure legends.

TABLE 5
All cGAS designs screened by the ISRE assay and their activity

| Variant | Active?* |
|---|---|
| K160D/R161P (CA-cGAS-04) | Yes |
| L159I/K160D/R161P | No |
| L159V/K160D/R161P | No |
| R158P/K160D/R161P | No |
| R158P/L159V/K160D/R161P | No |
| R158P/K160D/R161P/I164Q/E166D | No |
| N196P/Y200F/H203Q/V204I | No |
| N196P/Y200V/H203K | No |
| N196P/T197I/Y200F/V204N | No |
| N196P/Y200F/V204F | No |
| T197V/Y200I/H203Q/V204F | No |
| T197I/Y200I/H203Q/V204I | No |
| Y200F/H203Q/V204I | No |
| Y200I/H203K/V204I | No |
| Y200F/H203K/V204I | No |
| T197L/Y200F/V203F | No |
| T197I/Y200F/V204I | No |
| N196L/Y200F/H203Q/V204I | No |
| N196L/Y200I/H203K/V204I | No |
| N196L/T197V/Y200F/H203K | No |
| N196L/Y200F/H203K | No |
| N196R/T197I/Y200I/H203K/V204I | No |
| N196F/T197L/Y200F/H203G/V204N | No |
| N196F/T197V/Y200I/H203K | No |
| N196F/T197L/Y200F/H203R | No |
| N196F/Y200F/V204I | No |
| K205L | No |
| R158P | No |
| K158P | No |
| K160P | No |
| R161P | No |
| N196W | No |
| N196F | No |
| Y200I | No |
| Y200F | No |
| K160D/R161P/N196P/T197V/Y200F/H203K (CA-cGAS-22) | Yes |
| K160D/R161P/T197I/Y200I/H203Q/V204I | No |
| K160D/R161P/Y200F/H203Q/V204I | No |
| K160D/R161P/Y200I/H203K/V204I | No |
| K160D/R161P/Y200F/H203K/V204I | No |
| K160D/R161P/Y200I/H203R/V204I | No |
| K160D/R161P/T197L/Y200F/V204F | No |
| K160D/R161P/Y200F/H203G/V204F | No |
| K160D/R161P/N196L/Y200F/H203Q/V204I | No |
| K160D/R161P/N196L/Y200I/H203K/V204I | No |
| K160D/R161P/N196L/T197V/Y200F/H203K (CA-cGAS-41) | Yes |
| K160D/R161P/N196L/Y200F/H203K (CA-cGAS-42) | Yes |
| K160D/R161P/N196R/Y200F/H203Q/V204I | No |
| K160D/R161P/N196R/Y200F/V204F | No |
| K160D/R161P/N196F/T197V/Y200I/H203K (CA-cGAS-50) | Yes |
| K160D/R161P/N196F/T197L/Y200F/H203R | No |
| K160D/R161P/K205L | No |
| K160D/R161P/N196W/I206V | No |

*Defined as above K395M/K399M baseline

TABLE 6
Sequences of CA-cGAS variants

>WT mcGAS
MEDPRRRTTAPRAKKPSAKRAPTQPSRTRAHAESCGPQRGARSRRAERDGDTTEKPRAPGPRVHPARATELTKDAQPSAMDA
AGATARPAVRVPQQQAILDPELPAVREPQPPADPEARKVVRGPSHRRGARSTGQPRAPRGSRKEPDKLKKVLDKLRLKRKDI
SEAAETVNKVVERLLRRMQKRESEFKGVEQLNTGSYYEHVKISAPNEFDVMFKLEVPRIELQEYYETGAFYLVKFKRIPRGN
PLSHFLEGEVLSATKMLSKFRKIIKEEVKEIKDIDVSVEKEKPGSPAVTLLIRNPEEISVDIILALESKGSWPISTKEGLPI
QGWLGTKVRTNLRREPFYLVPKNAKDGNSFQGETWRLSFSHTEKYILNNHGIEKTCCESSGAKCCRKECLKLMKYLLEQLKK
EFQELDAFCSYHVKTAIFHMWTQDPQDSQWDPRNLSSCFDKLLAFFLECLRTEKLDHYFIPKFNLFSQELIDRKSKEFLSKK
IEYERNNGFPIFDKL (SEQ ID NO: 61)
>d147 WT mcGAS
SPDKLKKVLDKLRLKRKDISEAAETVNKVVERLLRRMQKRESEFKGVEQLNTGSYYEHVKISAPNEFDVMFKLEVPRIELQE
YYETGAFYLVKFKRIPRGNPLSHFLEGEVLSATKMLSKFRKIIKEEVKEIKDIDVSVEKEKPGSPAVTLLIRNPEEISVDII
LALESKGSWPISTKEGLPIQGWLGTKVRTNLRREPFYLVPKNAKDGNSFQGETWRLSFSHTEKYILNNHGIEKTCCESSGAK
CCRKECLKLMKYLLEQLKKEFQELDAFCSYHVKTAIFHMWTQDPQDSQWDPRNLSSCFDKLLAFFLECLRTEKLDHYFIPKF
NLFSQELIDRKSKEFLSKKIEYERNNGFPIFDKL (SEQ ID NO: 62)
>WT hcGAS
MQPWHGKAMQRASEAGATAPKASARNARGAPMDPTESPAAPEAALPKAGKFGPARKSGSRQKKSAPDTQERPPVRATGARAK
KAPQRAQDTQPSDATSAPGAEGLEPPAAREPALSRAGSCRQRGARCSTKPRPPPGPWDVPSPGLPVSAPILVRRDAAPGASK
LRAVLEKLKLSRDDISTAAGMVKGVVDHLLLRLKCDSAFRGVGLLNTGSYYEHVKISAPNEFDVMFKLEVPRIQLEEYSNTR
AYYFVKFKRNPKENPLSQFLEGEILSASKMLSKFRKIIKEEINDIKDTDVIMKRKRGGSPAVTLLISEKISVDITLALESKS
SWPASTQEGLRIQNWLSAKVRKQLRLKPFYLVPKHAKEGNGFQEETWRLSFSHIEKEILNNHGKSKTCCENKEEKCCRKDCL
KLMKYLLEQLKERFKDKKHLDKFSSYHVKTAFFHVCTQNPQDSQWDRKDLGLCFDNCVTYFLQCLRTEKLENYFIPEFNLES
SNLIDKRSKEFLTKQIEYERNNEFPVFDEF (SEQ ID NO: 63)
>K395M/K399M mcGAS
MEDPRRRTTAPRAKKPSAKRAPTQPSRTRAHAESCGPQRGARSRRAERDGDTTEKPRAPGPRVHPARATELTKDAQPSAMDA
AGATARPAVRVPQQQAILDPELPAVREPQPPADPEARKVVRGPSHRRGARSTGQPRAPRGSRKEPDKLKKVLDKLRLKRKDI
SEAAETVNKVVERLLRRMQKRESEFKGVEQLNTGSYYEHVKISAPNEFDVMFKLEVPRIELQEYYETGAFYLVKFKRIPRGN
PLSHFLEGEVLSATKMLSKFRKIIKEEVKEIKDIDVSVEKEKPGSPAVTLLIRNPEEISVDIILALESKGSWPISTKEGLPI
QGWLGTKVRTNLRREPFYLVPKNAKDGNSFQGETWRLSFSHTEKYILNNHGIEKTCCESSGAKCCRMECLMLMKYLLEQLKK
EFQELDAFCSYHVKTAIFHMWTQDPQDSQWDPRNLSSCFDKLLAFFLECLRTEKLDHYFIPKFNLFSQELIDRKSKEFLSKK
IEYERNNGFPIFDKL (SEQ ID NO: 64)
>d147 K395M/K399M mcGAS
SPDKLKKVLDKLRLKRKDISEAAETVNKVVERLLRRMQKRESEFKGVEQLNTGSYYEHVKISAPNEFDVMFKLEVPRIELQE
YYETGAFYLVKFKRIPRGNPLSHFLEGEVLSATKMLSKFRKIIKEEVKEIKDIDVSVEKEKPGSPAVTLLIRNPEEISVDII
LALESKGSWPISTKEGLPIQGWLGTKVRTNLRREPFYLVPKNAKDGNSFQGETWRLSFSHTEKYILNNHGIEKTCCESSGAK
CCRMECLMLMKYLLEQLKKEFQELDAFCSYHVKTAIFHMWTQDPQDSQWDPRNLSSCFDKLLAFFLECLRTEKLDHYFIPKF
NLFSQELIDRKSKEFLSKKIEYERNNGFPIFDKL (SEQ ID NO: 65)
>K395M/K399M hcGAS
MQPWHGKAMQRASEAGATAPKASARNARGAPMDPTESPAAPEAALPKAGKFGPARKSGSRQKKSAPDTQERPPVRATGARAK
KAPQRAQDTQPSDATSAPGAEGLEPPAAREPALSRAGSCRQRGARCSTKPRPPPGPWDVPSPGLPVSAPILVRRDAAPGASK
LRAVLEKLKLSRDDISTAAGMVKGVVDHLLLRLKCDSAFRGVGLLNTGSYYEHVKISAPNEFDVMFKLEVPRIQLEEYSNTR
AYYFVKFKRNPKENPLSQFLEGEILSASKMLSKFRKIIKEEINDIKDTDVIMKRKRGGSPAVTLLISEKISVDITLALESKS
SWPASTQEGLRIQNWLSAKVRKQLRLKPFYLVPKHAKEGNGFQEETWRLSFSHIEKEILNNHGKSKTCCENKEEKCCRMDCL
MLMKYLLEQLKERFKDKKHLDKFSSYHVKTAFFHVCTQNPQDSQWDRKDLGLCFDNCVTYFLQCLRTEKLENYFIPEFNLFS
SNLIDKRSKEFLTKQIEYERNNEFPVFDEF (SEQ ID NO: 66)
>K395A/K399A mcGAS
MEDPRRRTTAPRAKKPSAKRAPTQPSRTRAHAESCGPQRGARSRRAERDGDTTEKPRAPGPRVHPARATELTKDAQPSAMDA
AGATARPAVRVPQQQAILDPELPAVREPQPPADPEARKVVRGPSHRRGARSTGQPRAPRGSRKEPDKLKKVLDKLRLKRKDI
SEAAETVNKVVERLLRRMQKRESEFKGVEQLNTGSYYEHVKISAPNEFDVMFKLEVPRIELQEYYETGAFYLVKFKRIPRGN
PLSHFLEGEVLSATKMLSKFRKIIKEEVKEIKDIDVSVEKEKPGSPAVTLLIRNPEEISVDIILALESKGSWPISTKEGLPI
QGWLGTKVRTNLRREPFYLVPKNAKDGNSFQGETWRLSFSHTEKYILNNHGIEKTCCESSGAKCCRAECLALMKYLLEQLKK
EFQELDAFCSYHVKTAIFHMWTQDPQDSQWDPRNLSSCFDKLLAFFLECLRTEKLDHYFIPKFNLFSQELIDRKSKEFLSKK
IEYERNNGFPIFDKL (SEQ ID NO: 67)
>CA-cGAS-04
MEDPRRRTTAPRAKKPSAKRAPTQPSRTRAHAESCGPQRGARSRRAERDGDTTEKPRAPGPRVHPARATELTKDAQPSAMDA
AGATARPAVRVPQQQAILDPELPAVREPQPPADPEARKVVRGPSHRRGARSTGQPRAPRGSRKEPDKLKKVLDKLRLDPKDI
SEAAETVNKVVERLLRRMQKRESEFKGVEQLNTGSYYEHVKISAPNEFDVMFKLEVPRIELQEYYETGAFYLVKFKRIPRGN
PLSHFLEGEVLSATKMLSKFRKIIKEEVKEIKDIDVSVEKEKPGSPAVTLLIRNPEEISVDIILALESKGSWPISTKEGLPI
QGWLGTKVRTNLRREPFYLVPKNAKDGNSFQGETWRLSFSHTEKYILNNHGIEKTCCESSGAKCCRMECLMLMKYLLEQLKK
EFQELDAFCSYHVKTAIFHMWTQDPQDSQWDPRNLSSCFDKLLAFFLECLRTEKLDHYFIPKFNLFSQELIDRKSKEFLSKK
IEYERNNGFPIFDKL (SEQ ID NO: 68)
>CA-cGAS-22
MEDPRRRTTAPRAKKPSAKRAPTQPSRTRAHAESCGPQRGARSRRAERDGDTTEKPRAPGPRVHPARATELTKDAQPSAMDA
AGATARPAVRVPQQQAILDPELPAVREPQPPADPEARKVVRGPSHRRGARSTGQPRAPRGSRKEPDKLKKVLDKLRLDPKDI
SEAAETVNKVVERLLRRMQKRESEFKGVEQLPVGSFYEKVKISAPNEFDVMFKLEVPRIELQEYYETGAFYLVKFKRIPRGN
PLSHFLEGEVLSATKMLSKFRKIIKEEVKEIKDIDVSVEKEKPGSPAVTLLIRNPEEISVDIILALESKGSWPISTKEGLPI
QGWLGTKVRTNLRREPFYLVPKNAKDGNSFQGETWRLSFSHTEKYILNNHGIEKTCCESSGAKCCRMECLMLMKYLLEQLKK
EFQELDAFCSYHVKTAIFHMWTQDPQDSQWDPRNLSSCFDKLLAFFLECLRTEKLDHYFIPKFNLFSQELIDRKSKEFLSKK
IEYERNNGFPIFDKL (SEQ ID NO: 69)
>d147 CA-cGAS-22
SPDKLKKVLDKLRLDPKDISEAAETVNKVVERLLRRMQKRESEFKGVEQLPVGSFYEKVKISAPNEFDVMFKLEVPRIELQE
YYETGAFYLVKFKRIPRGNPLSHFLEGEVLSATKMLSKFRKIIKEEVKEIKDIDVSVEKEKPGSPAVTLLIRNPEEISVDII
LALESKGSWPISTKEGLPIQGWLGTKVRTNLRREPFYLVPKNAKDGNSFQGETWRLSFSHTEKYILNNHGIEKTCCESSGAK
CCRMECLMLMKYLLEQLKKEFQELDAFCSYHVKTAIFHMWTQDPQDSQWDPRNLSSCFDKLLAFFLECLRTEKLDHYFIPKF
NLFSQELIDRKSKEFLSKKIEYERNNGFPIFDKL (SEQ ID NO: 70)
>CA-cGAS-22 (human)
MQPWHGKAMQRASEAGATAPKASARNARGAPMDPTESPAAPEAALPKAGKFGPARKSGSRQKKSAPDTQERPPVRATGARAK
KAPQRAQDTQPSDATSAPGAEGLEPPAAREPALSRAGSCRQRGARCSTKPRPPPGPWDVPSPGLPVSAPILVRRDAAPGASK
LRAVLEKLKLDPDDISTAAGMVKGVVDHLLLRLKCDSAFRGVGLLPVGSFYEKVKISAPNEFDVMFKLEVPRIQLEEYSNTR
AYYFVKFKRNPKENPLSQFLEGEILSASKMLSKFRKIIKEEINDIKDTDVIMKRKRGGSPAVTLLISEKISVDITLALESKS
SWPASTQEGLRIQNWLSAKVRKQLRLKPFYLVPKHAKEGNGFQEETWRLSFSHIEKEILNNHGKSKTCCENKEEKCCRKDCL
KLMKYLLEQLKERFKDKKHLDKFSSYHVKTAFFHVCTQNPQDSQWDRKDLGLCFDNCVTYFLQCLRTEKLENYFIPEENLFS
SNLIDKRSKEFLTKQIEYERNNEFPVFDEF (SEQ ID NO: 71)
>CA-cGAS-41
MEDPRRRTTAPRAKKPSAKRAPTQPSRTRAHAESCGPQRGARSRRAERDGDTTEKPRAPGPRVHPARATELTKDAQPSAMDA
AGATARPAVRVPQQQAILDPELPAVREPQPPADPEARKVVRGPSHRRGARSTGQPRAPRGSRKEPDKLKKVLDKLRLDPKDI
SEAAETVNKVVERLLRRMQKRESEFKGVEQLLVGSFYEKVKISAPNEFDVMFKLEVPRIELQEYYETGAFYLVKFKRIPRGN
PLSHFLEGEVLSATKMLSKFRKIIKEEVKEIKDIDVSVEKEKPGSPAVTLLIRNPEEISVDIILALESKGSWPISTKEGLPI
QGWLGTKVRTNLRREPFYLVPKNAKDGNSFQGETWRLSFSHTEKYILNNHGIEKTCCESSGAKCCRMECLMLMKYLLEQLKK
EFQELDAFCSYHVKTAIFHMWTQDPQDSQWDPRNLSSCFDKLLAFFLECLRTEKLDHYFIPKFNLFSQELIDRKSKEFLSKK
IEYERNNGFPIFDKL (SEQ ID NO: 72)
>d147 CA-cGAS-41
SPDKLKKVLDKLRLDPKDISEAAETVNKVVERLLRRMQKRESEFKGVEQLLVGSFYEKVKISAPNEFDVMFKLEVPRIELQE
YYETGAFYLVKFKRIPRGNPLSHFLEGEVLSATKMLSKFRKIIKEEVKEIKDIDVSVEKEKPGSPAVTLLIRNPEEISVDII
LALESKGSWPISTKEGLPIQGWLGTKVRTNLRREPFYLVPKNAKDGNSFQGETWRLSFSHTEKYILNNHGIEKTCCESSGAK
CCRMECLMLMKYLLEQLKKEFQELDAFCSYHVKTAIFHMWTQDPQDSQWDPRNLSSCFDKLLAFFLECLRTEKLDHYFIPKF
NLFSQELIDRKSKEFLSKKIEYERNNGFPIFDKL (SEQ ID NO: 73)
>CA-cGAS-41 (human)
MQPWHGKAMQRASEAGATAPKASARNARGAPMDPTESPAAPEAALPKAGKFGPARKSGSRQKKSAPDTQERPPVRATGARAK
KAPQRAQDTQPSDATSAPGAEGLEPPAAREPALSRAGSCRQRGARCSTKPRPPPGPWDVPSPGLPVSAPILVRRDAAPGASK
LRAVLEKLKLDPDDISTAAGMVKGVVDHLLLRLKCDSAFRGVGLLLVGSFYEKVKISAPNEFDVMFKLEVPRIQLEEYSNTR
AYYFVKFKRNPKENPLSQFLEGEILSASKMLSKFRKIIKEEINDIKDTDVIMKRKRGGSPAVTLLISEKISVDITLALESKS
SWPASTQEGLRIQNWLSAKVRKQLRLKPFYLVPKHAKEGNGFQEETWRLSFSHIEKEILNNHGKSKTCCENKEEKCCRKDCL
KLMKYLLEQLKERFKDKKHLDKESSYHVKTAFFHVCTQNPQDSQWDRKDLGLCFDNCVTYFLQCLRTEKLENYFIPEFNLFS
SNLIDKRSKEFLTKQIEYERNNEFPVFDEF (SEQ ID NO: 74)
>CA-cGAS-42
MEDPRRRTTAPRAKKPSAKRAPTQPSRTRAHAESCGPQRGARSRRAERDGDTTEKPRAPGPRVHPARATELTKDAQPSAMDA
AGATARPAVRVPQQQAILDPELPAVREPQPPADPEARKVVRGPSHRRGARSTGQPRAPRGSRKEPDKLKKVLDKLRLDPKDI
SEAAETVNKVVERLLRRMQKRESEFKGVEQLLTGSFYEKVKISAPNEFDVMFKLEVPRIELQEYYETGAFYLVKFKRIPRGN
PLSHFLEGEVLSATKMLSKFRKIIKEEVKEIKDIDVSVEKEKPGSPAVTLLIRNPEEISVDIILALESKGSWPISTKEGLPI
QGWLGTKVRTNLRREPFYLVPKNAKDGNSFQGETWRLSFSHTEKYILNNHGIEKTCCESSGAKCCRMECLMLMKYLLEQLKK
EFQELDAFCSYHVKTAIFHMWTQDPQDSQWDPRNLSSCFDKLLAFFLECLRTEKLDHYFIPKFNLFSQELIDRKSKEFLSKK
IEYERNNGFPIFDKL (SEQ ID NO: 75)
>d147 CA-cGAS-42
SPDKLKKVLDKLRLDPKDISEAAETVNKVVERLLRRMQKRESEFKGVEQLLTGSFYEKVKISAPNEFDVMFKLEVPRIELQE
YYETGAFYLVKFKRIPRGNPLSHFLEGEVLSATKMLSKFRKIIKEEVKEIKDIDVSVEKEKPGSPAVTLLIRNPEEISVDII
LALESKGSWPISTKEGLPIQGWLGTKVRTNLRREPFYLVPKNAKDGNSFQGETWRLSFSHTEKYILNNHGIEKTCCESSGAK
CCRMECLMLMKYLLEQLKKEFQELDAFCSYHVKTAIFHMWTQDPQDSQWDPRNLSSCFDKLLAFFLECLRTEKLDHYFIPKF
NLFSQELIDRKSKEFLSKKIEYERNNGFPIFDKL (SEQ ID NO: 76)
>CA-cGAS-42 (human)
MQPWHGKAMQRASEAGATAPKASARNARGAPMDPTESPAAPEAALPKAGKFGPARKSGSRQKKSAPDTQERPPVRATGARAK
KAPQRAQDTQPSDATSAPGAEGLEPPAAREPALSRAGSCRQRGARCSTKPRPPPGPWDVPSPGLPVSAPILVRRDAAPGASK
LRAVLEKLKLDPDDISTAAGMVKGVVDHLLLRLKCDSAFRGVGLLLTGSFYEKVKISAPNEFDVMFKLEVPRIQLEEYSNTR
AYYFVKFKRNPKENPLSQFLEGEILSASKMLSKGRKIIKEEINDIKDTDVIMKRKRGGSPAVTLLISEKISVDITLALESKS
SWPASTQEGLRIQNWLSAKVRKQLRLKPFYLVPKHAKEGNGFQEETWRLSFSHIEKEILNNHGKSKTCCENKEEKCCRKDCL
KLMKYLLEQLKERFKDKKHLDKFSSYHVKTAFFHVCTQNPQDSQWDRKDLGLCFDNCVTYFLQCLRTEKLENYFIPEFNLFS
SNLIDKRSKEFLTKQIEYERNNEFPVFDEF (SEQ ID NO: 77)
>CA-cGAS-50
MEDPRRRTTAPRAKKPSAKRAPTQPSRTRAHAESCGPQRGARSRRAERDGDTTEKPRAPGPRVHPARATELTKDAQPSAMDA
AGATARPAVRVPQQQAILDPELPAVREPQPPADPEARKVVRGPSHRRGARSTGQPRAPRGSRKEPDKLKKVLDKLRLDPKDI
SEAAETVNKVVERLLRRMQKRESEFKGVEQLFVGSIYEKVKISAPNEFDVMFKLEVPRIELQEYYETGAFYLVKFKRIPRGN
PLSHFLEGEVLSATKMLSKFRKIIKEEVKEIKDIDVSVEKEKPGSPAVTLLIRNPEEISVDIILALESKGSWPISTKEGLPI
QGWLGTKVRTNLRREPFYLVPKNAKDGNSFQGETWRLSFSHTEKYILNNHGIEKTCCESSGAKCCRMECLMLMKYLLEQLKK
EFQELDAFCSYHVKTAIFHMWTQDPQDSQWDPRNLSSCFDKLLAFFLECLRTEKLDHYFIPKFNLFSQELIDRKSKEFLSKK
IEYERNNGFPIFDKL (SEQ ID NO: 78)
>d147 CA-cGAS-50
SPDKLKKVLDKLRLDPKDISEAAETVNKVVERLLRRMQKRESEFKGVEQLFVGSIYEKVKISAPNEFDVMFKLEVPRIELQE
YYETGAFYLVKFKRIPRGNPLSHFLEGEVLSATKMLSKFRKIIKEEVKEIKDIDVSVEKEKPGSPAVTLLIRNPEEISVDII
LALESKGSWPISTKEGLPIQGWLGTKVRTNLRREPFYLVPKNAKDGNSFQGETWRLSFSHTEKYILNNHGIEKTCCESSGAK
CCRMECLMLMKYLLEQLKKEFQELDAFCSYHVKTAIFHMWTQDPQDSQWDPRNLSSCFDKLLAFFLECLRTEKLDHYFIPKF
NLFSQELIDRKSKEFLSKKIEYERNNGFPIFDKL (SEQ ID NO: 79)
>CA-cGAS-50 (human)
MQPWHGKAMQRASEAGATAPKASARNARGAPMDPTESPAAPEAALPKAGKFGPARKSGSRQKKSAPDTQERPPVRATGARAK
KAPQRAQDTQPSDATSAPGAEGLEPPAAREPALSRAGSCRQRGARCSTKPRPPPGPWDVPSPGLPVSAPILVRRDAAPGASK
LRAVLEKLKLDPDDISTAAGMVKGVVDHLLLRLKCDSAFRGVGLLFVGSIYEKVKISAPNEFDVMFKLEVPRIQLEEYSNTR
AYYFVKFKRNPKENPLSQFLEGEILSASKMLSKFRKIIKEEINDIKDTDVIMKRKRGGSPAVTLLISEKISVDITLALESKS
SWPASTQEGLRIQNWLSAKVRKQLRLKPFYLVPKHAKEGNGFQEETWRLSFSHIEKEILNNHGKSKTCCENKEEKCCRKDCL
KLMKYLLEQLKERFKDKKHLDKFSSYHVKTAFFHVCTQNPQDSQWDRKDLGLCFDNCVTYFLQCLRTEKLENYFIPEFNLFS
SNLIDKRSKEFLTKQIEYERNNEFPVFDEF (SEQ ID NO: 80)

1. A polypeptide comprising an amino acid sequence at least 20% identical to the amino acid sequence of SEQ ID NO:1 or 2,
SEQ ID NO: 1
(DAAP)GASKLRAVLEKLKLSRDDISTAAGMVKGVVDHLLLRLKCDSAFR
GVGLLNTGSYYEHVKISAPNEFDVMFKLEVPRIQLEEYSNTRAYYFVKFK
RNPKENPLSQFLEGEILSASKMLSKFRKIIKEEINDIKDTDVIMKRKRGG
SPAVTLLISEKISVDITLALESKSSWPASTQEGLRIQNWLSAKVRKQLRL
KPFYLVPKHAKEGNGFQEETWRLSFSHIEKEILNNHGKSKTCCENKEEKC
CRKDCLKLMKYLLEQLKERFKDKKHLDKFSSYHVKTAFFHVCTQNPQDSQ
WDRKDLGLCFDNCVTYFLQCLRTEKLENYFIPEFNLFSSNLIDKRSKEFL
TKQIEYERNNEFPVFDEF
(SEQ ID NO: 2)
(M)QPWHGKAMQRASEAGATAPKASARNARGAPMDPTESPAAPEAALPKA
GKFGPARKSGSRQKKSAPDTQERPPVRATGARAKKAPQRAQDTQPSDATS
APGAEGLEPPAAREPALSRAGSCRQRGARCSTKPRPPPGPWDVPSPGLPV
SAPILVRRDAAPGASKLRAVLEKLKLSRDDISTAAGMVKGVVDHLLLRLK
CDSAFRGVGLLNTGSYYEHVKISAPNEFDVMFKLEVPRIQLEEYSNTRAY
YFVKFKRNPKENPLSQFLEGEILSASKMLSKFRKIIKEEINDIKDTDVIM
KRKRGGSPAVTLLISEKISVDITLALESKSSWPASTQEGLRIQNWLSAKV
RKQLRLKPFYLVPKHAKEGNGFQEETWRLSFSHIEKEILNNHGKSKTCCE
NKEEKCCRKDCLKLMKYLLEQLKERFKDKKHLDKFSSYHVKTAFFHVCTQ
NPQDSQWDRKDLGLCFDNCVTYFLWCLRTEKLENYFIPEFNLFSSNLIDK
RSKEFLTKQIEYERNNEFPVFDEF;
wherein the polypeptide is a constitutively active Cyclic GMP-AMP Synthase (cGAS) mutant;
wherein residues in parentheses are optional and may be present or absent;
wherein relative to residue numbering in SEQ ID NO:1:
residue 56 is G, residue 57 is S, residue 60 is E, residue 69 is E, and residue 71 is D; and
wherein one or both of the following are true:
(a) residue 19 is P and residue 20 is A; or residue 19 is D and residue 20 is P; and
(b) residue 54 is P, L, or F and residue 58 is F, I, W, or L.

2. The polypeptide of claim 1, wherein residue 19 is P and residue 20 is relative to residue numbering in SEQ ID NO:1.

3. The polypeptide of claim 1, wherein residue 19 is D and residue 20 is P relative to residue numbering in SEQ ID NO:1.

4. The polypeptide of claim 1, wherein residue 54 is P, L, or F and residue 58 is F, I, W, or L relative to residue numbering in SEQ ID NO:1.

5. The polypeptide of claim 1, wherein residue 55 is V, I, A, S, or G relative to residue numbering in SEQ ID NO:1, preferably wherein residue 55 is V or I, most preferably wherein residue 55 is V.

6. The polypeptide of claim 1, wherein residue 61 is K relative to residue numbering in SEQ ID NO:1.

7. The polypeptide of claim 1, wherein 1, 2, or all 3 of the following are true: residue 62 is V, residue 63 is K, and/or residue 64 is I, relative to residue numbering in SEQ ID NO:1.

8. The polypeptide of claim 1, wherein one or more of the following is true relative to residue numbering in SEQ ID NO:1:
residue 17 is S;
residue 18 is I;
residue 59 is E, F, W, L, or I; preferably wherein residue 59 is F, W, L, or I, more preferably wherein residue 59 is F or W;
residue 65 is G or V;
residue 251 is M; and/or
residue 255 is M.

9. A polypeptide comprising an amino acid sequence at least 20% identical to the amino acid sequence of SEQ ID NO:3 or 4,
(SEQ ID NO: 3)
(SPD)KLKKVLDKLRLKRKDISEAAETVNKVVERLLRRMQKRESEEKGVE
QLNTGSYYEHVKISAPNEFDVMFKLEVPRIELQEYYETGAFYLVKFKRIP
RGNPLSHFLEGEVLSATKMLSKFRKIIKEEVKEIKDIDVSVEKEKPGSPA
VTLLIRNPEEISVDIILALESKGSWPISTKEGLPIQGWLGTKVRTNLRRE
PFYLVPKNAKDGNSFQGETWRLSFSHTEKYILNNHGIEKTCCESSGAKCC
RKECLKIMKYLLEQLKKEFQELDAFCSYHVKTAIFHMWTQDPQDSQWDPR
NLSSCFDKLLAFFLECLRTEKLDHYFIPKFNLFSQELIDRKSKEFLSKKI
EYERNNGFPIFDKL
(SEQ ID NO: 4)
(M)EDPRRRTTAPRAKKPSAKRAPTQPSRTRAHAESCGPQRGARSRRAER
DGDTTEKPRAPGPRVHPARATELTKDAQPSAMDAAGATARPAVRVPQQQA
ILDPELPAVREPQPPADPEARKVVRGPSHRRGARSTGQPRAPRGSRKEPD
KLKKVLDKLRLKRKDISEAAETVNKVVERLLRRMQKRESEFKGVEQLNTG
SYYEHVKISAPNEFDVMFKLEVPRIELQEYYETGAFYLVKFKRIPRGNPL
SHFLEGEVLSATKMLSKFRKIIKEEVKEIKDIDVSVEKEKPGSPAVTLLI
RNPEEISVDIILALESKGSWPISTKEGLPIQGWLGTKVRTNLRREPFYLV
PKNAKDGNSFQGETWRLSFSHTEKYILNNHGIEKTCCESSGAKCCRKECL
KMKYLLEQLKKEFQELDAFCSYHVKTAIFHMWTQDPQDSQWDPRNLSSCF
DKLLAFFLECLRTEKLDHYFIPKENLFSQELIDRKSKEFLSKKIEYERNN
GFPIFDKL
wherein the polypeptide is a constitutively active Cyclic GMP-AMP Synthase (cGAS) mutant;
wherein residues in parentheses are optional and may be present or absent;
wherein relative to residue numbering in SEQ ID NO:3:
residue 53 is G, residue 54 is S, residue 57 is E, residue 66 is E, and residue 68 is D; and
wherein one or both of the following are true:
(a) residue 15 is P and residue 16 is A; or residue 15 is D and residue 16 is P; and
(b) residue 51 is P, L, or F and residue 55 is F, I, W, or L.

10. The polypeptide of claim 9, wherein residue 15 is P and residue 16 is A relative to residue numbering in SEQ ID NO:3.

11. The polypeptide of claim 9, wherein residue 15 is D and residue 16 is P relative to residue numbering in SEQ ID NO:3.

12. The polypeptide of claim 9, wherein residue 51 is P, L, or F and residue 55 is F, I, W, or L, relative to residue numbering in SEQ ID NO:3.

13. The polypeptide of claim 9, wherein residue 52 is V, I, A, G, or S relative to residue numbering in SEQ ID NO:3; preferably wherein residue 52 is V or I, or more preferably wherein residue 52 is V.

14. The polypeptide of claim 9, wherein residue 58 is K relative to residue numbering in SEQ ID NO:3.

15. The polypeptide of claim 9, wherein 1, 2, or all 3 are true: residue 59 is V, residue 60 is K, and/or residue 61 is I relative to residue numbering in SEQ ID NO:3.

16. The polypeptide of claim 10, wherein one or more of the following is true relative to residue numbering in SEQ ID NO:3:
residue 13 is S;
residue 14 is I;
residue 56 is E, F, W, L, or I; preferably wherein residue 56 is F, W, L, or I, more preferably wherein residue 56 is F ow W;
residue 62 is G or V;
residue 250 is M;
residue 254 is M.

17. The polypeptide of claim 1, wherein the polypeptide does not include the mutation or combination of mutations listed on a single line in Table 1 relative to residue numbering in SEQ ID NO:1, in Table 2 relative to residue numbering in SEQ ID NO:2, in Table 3 relative to residue numbering in SEQ ID NO:3, or in Table 4 relative to residue numbering in SEQ ID NO:4.

18. The polypeptide of claim 1, comprising an amino acid sequence at least 20% identical to the amino acid sequence selected from the group consisting of SEQ ID NOS:5-59.

19.-20. (canceled)

21. A pharmaceutical composition comprising the polypeptide of claim 1, and a pharmaceutically acceptable carrier.

22. A nucleic acid encoding the polypeptide of claim 1.

23.-28. (canceled)
